(19) United States
(12) Patent Application Publication
(10) Pub. No.: US 2010/0298238 A1
(43) Pub. Date: Nov. 25, 2010

(54) NONTOXIC SHIGA-LIKE TOXIN MUTANT COMPOSITIONS AND METHODS

(75) Inventors: Nilgun E. Tumer, Belle Mead, NJ (US); Rong Di, East Brunswick, NJ (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: RUTGERS, THE STATE UNIVERSITY, New Brunswick, NJ (US)

(21) Appl. No.: 12/682,104

(22) PCT Filed: Oct. 8, 2008
(86) PCT No.: PCT/US08/11631

§ 371 Date: Jul. 28, 2010

Related U.S. Application Data
(60) Provisional application No. 60/978,280, filed on Oct. 8, 2007.

(51) Int. Cl. A61K 38/16; C07K 14/25; C07H 21/04; C12N 15/70; C12N 1/21; A61P 31/04; A61P 7/00

(52) U.S. Cl. 514/21.2; 530/350; 536/23.7; 435/320.1; 435/252.33; 514/21.3; 514/21.4

(57) ABSTRACT

Disclosed are nontoxic mutants of Shiga-like toxin (Stx1 or Stx2), nucleic acids encoding them, compositions containing the mutants and methods of using the mutants in connection with hemolytic euremic syndrome (HUS). Also disclosed are methods of treating HUS using L3 protein fragments, the nontoxic Stx1 or Stx2 mutants, or combinations thereof.

### CROSS-REFERENCE TO RELATED APPLICATIONS

The present application claims the benefit of U.S. Provisional Application Ser. No. 60/978,280, filed Oct. 8, 2007, entitled, “Nontoxic Shiga-Like Toxin Mutant Compositions and Methods”, the disclosure of which is hereby incorporated herein by reference.

### STATEMENT REGARDING FEDERALLY SPONSORED RESEARCH

The development of this invention was supported by National Institutes of Health grant A1068869. Thus, the Government may have rights in the invention.

### BACKGROUND OF THE INVENTION

Infections with Shigella dysenteriae, which produces Shiga toxin, and the diarrheagenic E. coli O157:H7, which produces Shiga-like toxins (Stx), are responsible for widespread disease and death. Bacteria producing Shiga-like toxin can survive in undercooked hamburger, milk, fruit juice and fresh produce. These bacteria are the most common cause of hemolytic euremic syndrome (HUS), a disease for which there is neither a vaccine nor an effective treatment.

Due to the relative ease of production and the lethality of Stx, Stx-producing E. coli is a major threat as an agent of bioterrorism and has been classified as a NIAID Category B Priority for biodefense. Recent deaths and illnesses due to Stx-producing E. coli O157:H7 in contaminated foods clearly illustrate the public health impact of these pathogens. HUS is the most common cause of renal failure in infants and young children in the United States.

E. coli O157:H7 produces genetically and antigenically distinct exotoxins designated Shiga-like toxin 1 (Stx1) and Shiga-like toxin 2 (Stx2), of which Stx2 is the primary virulence factor for HUS. There are no effective treatment measures and no therapeutics effective against Stx-mediated HUS, largely due to the lack of animal models that reproduce HUS. Since antibiotic treatment has not been shown to alter clinical outcome and is not recommended, development of therapeutics for Stx-mediated cytotoxicity has become an important public health priority.

### SUMMARY OF THE INVENTION

A first aspect of the present invention is directed to an isolated nontoxic mutant comprising an A1 subunit of Shiga-like toxin, wherein said mutant differs from a wild-type subunit A1 of Shiga-like toxin 1 (Stx1), designated by SEQ ID NO:6, in terms of one or more amino acid substitutions, or wherein said mutant differs from a wild-type subunit A1 of Shiga-like toxin 2 (Stx2), designated as SEQ ID NO:8, in terms of one or more amino acid substitutions, except (1-297, E167/R170A), and/or which lacks from about 9 to 193 C-terminal residues thereof. DNAs, constructs (e.g., vectors containing the DNAs) and non-human hosts (e.g., bacterial, yeast, plant or mammalian cells) containing the DNAs, are also provided.

Another aspect of the present invention is directed to a composition containing the mutant and a carrier.

Another aspect of the present invention is directed to a method of treating an individual at risk of exposure to E. coli 0157:H7 or suspected of having hemolytic euremic syndrome (HUS), comprising administering to an individual in need thereof an effective amount of a nontoxic mutant of Stx1 or Stx2, wherein said mutant comprises a non-wild-type A1 subunit.

Yet another aspect of the present invention is directed to a method of treating hemolytic euremic syndrome (HUS) infection comprising administering to a human in need thereof an effective amount of a nontoxic mutant of Stx1 or Stx2, wherein said mutant comprises a non-wild-type A1 subunit, an effective amount of a eukaryotic L3 protein or fragment thereof containing from 21 to about 100 N-terminal amino acids thereof, or effective amounts of both said nontoxic mutant and said L3 fragment.

In another aspect of the present invention is directed to a method of treating hemolytic euremic syndrome (HUS) infection comprising administering to a human in need thereof an effective amount of an eukaryotic L3 protein or fragment thereof containing from 21 to about 100 N-terminal amino acids.

Applicants have identified nontoxic mutant forms of Stx1 and Stx2. Currently, there is no approved antidote, vaccine or other specific medical treatment option for exposure to Stxs. The nontoxic mutants are thus useful in developing vaccines or other therapeutic treatment measures against infections mediated by Stx-producing microorganisms such as E. coli O157:H7. Accordingly, the present invention provides means for combating Stx-associated HUS and for counteracting the potential use of these toxins as agents of bioterror.

### BRIEF DESCRIPTION OF THE DRAWINGS

FIGS. 1A-C show the viability of yeast cells expressing Stx1A and point mutants. Yeast cells were first grown in SD-Ura medium containing glucose to OD600 of 0.3 and then transferred to SD-Ura supplemental with galactose. At 0-hr (top panel) and 10-hr (bottom panel) post-induction, cells were spotted on SD-Ura plates containing glucose.

FIG. 2 shows immunoblot analysis of Stx1A and mutants' expression. The blot was probed with anti-V5 monoclonal antibody to determine the expression level of wt Stx1 and mutants. The blot was then stripped with guanidine hydrochloride and probed with anti-Dpm1 monoclonal antibody to show the equal loading of proteins.

FIG. 3 shows In vivo depurination of rRNA in yeast cells expressing wt Stx1 and mutants. Total RNA was isolated and subjected to the dual primer extension analysis using the depurination (Dep) primer to measure the extent of depurination oand the 25S rRNA primer (25S) to measure the total amount of 25S rRNA.

FIGS. 4A-C show the viability of yeast cells expressing Stx2A and point mutants. Yeast cells were first grown in SD-Ura medium containing glucose to OD600 of 0.3 and then transferred to SD-Ura supplemental with galactose. At 0-hr (top panel) and 10-hr (bottom panel) post-induction, cells were spotted on SD-Ura plates containing glucose.

FIG. 5 shows In vivo depurination of rRNA in yeast cells expressing wt Stx1 and mutants. Total RNA was isolated and subjected to the dual primer extension analysis using the depurination (Dep) primer to measure the extent of depurination oand the 25S rRNA primer (25S) to measure the total amount of 25S rRNA.

FIG. 6 shows viability of wild type yeast cells expressing STX1A alone, Stx1A together with L3Δ99 (Stx1A/L3Δ99) or harboring the empty vector at indicated hours (left) after induction. The panel on the right shows the viability of mak8-1 cells expressing Stx1A.

FIG. 7 shows viability of wild type yeast cells expressing STX2A alone, Stx1A together with L3Δ99 (Stx2A/L3Δ99) or harboring the empty vector at indicated hours (left) after induction. The panel on the right shows the viability of mak8-1 cells expressing Stx1A.

FIG. 8 shows ribosome depurination using dual primer extension analysis 6 hr after induction of cells expressing Stx1A and Stx2A alone or together with L3Δ99.

FIG. 9 shows ribosome depurination using dual primer extension analysis 6 hr after induction of wild type or mak8-1 cells expressing Stx1A or Stx2A.

FIG. 10 shows viability of yeast co-expressing Stx1A or Stx2A and wild type or mutant forms of L3Δ99. L3Δ99 RNA produces RNA, but not protein, while L3Δ21 contains only the first 21 amino acids of L3.

FIG. 11 shows vero cell ribosome depurination using dual primer extension 24 hr after transfection with Stx1 or Stx2 alone or together with L3Δ99 cloned in pcDNA3.1(+)

### DETAILED DESCRIPTION

As contemplated herein this invention embodies mutant forms of shiga and shiga-like toxins and their uses to treat bacterial infections, e.g., infections with. Shigella dysenteriae, producing Shiga toxin, and Diarrheagenic E. coli producing Shiga-like toxins (Stx). These infections include but are not limited to hemolytic uremic syndrome (HUS). It is to be understood that the present invention includes methods of treating individuals suffering from any infection with a virulent strain of an enterohemorragic E. coli (EHEC) including E. coli strains 931, 3100-85, and 933 or Shiga toxin producing E. coli (STEC).

It is contemplated that the present invention embodies a method of treating an individual at risk of exposure to E. coli 0157:H7, or suspected of having hemolytic uremic syndrome (HUS), comprising administering to an individual in need thereof an effective amount of a nontoxic mutant of Stx1 or Stx2, wherein said mutant comprises a non-wild-type A1 subunit. Such individuals may have been exposed to the bacteria, but have not had a confirmatory diagnosis of infection. Thus, the mutant functions as a vaccine.

It is further contemplated that the present invention embodies a method of treating hemolytic euremic syndrome (HUS) infection comprising administering to a human in need thereof an effective amount of a nontoxic mutant of Stx1 or Stx2, wherein said mutant comprises a non-wild-type A1 subunit, an effective amount of a fragment of a eukaryotic L3 protein containing from 21 to about 100 N-terminal amino acids thereof, or effective amounts of both said nontoxic mutant and said L3 fragment.

The bacterial infections associated with Shigella dysenteriae may be characterized by the production of ribosomal inactivating proteins (RIP), e.g., pokeweed antiviral protein (PAP), ricin, shiga toxin, and shiga-like toxin. The. Shiga-like toxin family contains two major, immunologically non-cross-reactive cytotoxins called Shiga-like toxin 1 (Stx1) and Shiga-like toxin 2 (Stx2) encoded by bacteriophage. Both Stx1 and Stx2 consist of an A catalytic subunit and pentamer of B subunits.

The A subunit of Stx1 is 293 amino acids in length. The A subunit of Stx2 is characterized as 297 amino acids in length. The A subunit may further be broken down into subunit 1 and subunit 2. There is discrepancy in the literature as to the length of the Stx1 A1 subunit as being 251 or 253 amino acids in length. See Garred et al., Furin-induced cleavage and activation of Shiga toxin. J. Biol. Chem. 270:10817-10821. (1995); LaPointe et al., A role for the protease-sensitive loop region of Shiga-like toxin 1 in the retrotranslocation of its A1 domain from the endoplasmic reticulum lumen. J. Biol. Chem. 280:23310-23318 (2005); Takao et al., Identity of molecular structure of Shiga-like toxin I (VT1) from Escherichia coli O157:H7 with that of Shiga toxin. Microbial pathogenesis 5:357-369 (1988). For purposes of this disclosure, however, the A1 subunit of Stx1 will be considered 251 amino acids in length. The A1 subunit of Stx2 is about 247 amino acids in length. The A2 subunits of Stx1 and Stx2 are formed by the remaining amino acids outside the A1 subunit.

Without being bound by any particular theory or mechanism of action, it is believed that the A subunit of Stx1 and Stx2 possesses RNA N-glycosidase activity that catalytically removes a specific adenine from the highly conserved sarcin/ricin loop (SRL) in the larger rRNA. This depurination event of the SRL prevents eukaryotic translation initiation and serves to block protein synthesis. Thus, it is believed that the A subunit is responsible for the toxicity associated with shiga-like toxins. As such, it is contemplated herein that non-toxic mutant forms of the Stx1 and Stx2 mutants disclosed herein have therapeutic uses in connection with infections the causative agent of which produces RIPs such as Shiga toxin and Stx1 and 2.

By the term “nontoxic”, it is meant that the mutants are less toxic to yeast cells than wild-type Stx1 or wild-type Stx2, i.e., they do not significantly inhibit cell growth (like wild-type Stx1 or Stx2) and they do not significantly affect cell viability. This determination can be made in accordance with a combination of standard techniques, illustrations of which are set forth in commonly owned United States Patent Application Publication 2004/0241673, which is hereby incorporated herein by reference, as well as in the working examples below.

By “wild-type Stx1A,” it is meant the mature Stx1 A subunit amino acid sequence 1-293, excluding the 22-amino acid N-terminal signal peptide (“the N-terminal signal sequence of wild-type Stx1”). Thus, by the term “wild-type,” or “Stx1A,” it is meant the Stx1 amino acid sequence 1-293 (hereinafter Stx1 (1-293)). The sequences designated SEQ ID NOS: 1 and 2 are the DNA and corresponding amino acid sequence of wild-type Stx1A:

SEQ ID NO: 1 - Stx1A nucleic acid sequence
SEQ ID NO: 2 - Stx1A amino acid sequence

| | |
|---|---|
| | 1 11 21 |
| | 31 41 51 61 |
| 1 | AAGGAATTTACCTTAGACTTCTCGACTGCAAAGACGTATGTAGATTCGCTGAATGTCATTCGCTCTGCAA |
| 1(+1) | K E F T L D F S T A K T |
| | Y V D S L N V I R S A |
| 71 | TAGGTACTCCATTACAGACTATTTCATCAGGAGGTACGTCTTTACTGATGATTGATAGTGGCTCAGGGGA |
| 24(+1) | I G T P L Q T I S S G G |
| | T S L L M I D S G S G D |
| 141 | TAATTTGTTTGCAGTTGATGTCAGAGGGATAGATCCAGAGGAAGGGCGGTTTAATAATCTACGGCTTATT |
| 48(+1) | N L F A V D V R G I D P |
| | E E G R F N N L R L I |
| 211 | GTTGAACGAAATAATTTATATGTGACAGGATTTGTTAACAGGACAAATAATGTTTTTTATCGCTTTGCTG |
| 71(+1) | V E R N N L Y V T G F V |
| | N R T N N V F Y R F A |
| 281 | ATTTTTCACATGTTACCTTTCCAGGTACAACAGCGGTTACATTGTCTGGTGACAGTAGCTATACCACGTT |
| 94(+1) | D F S H V T F P G T T A V |
| | T L S G D S S Y T T L |
| 351 | ACAGCGTGTTGCAGGGATCAGTCGTACGGGGATGCAGATAAATCGCCATTCGTTGACTACTTCTTATCTG |
| 118(+1) | Q R V A G I S R T G M |
| | Q I N R H S L T T S Y L |
| 421 | GATTTAATGTCGCATAGTGGAACCTCACTGACGCAGTCTGTGGCAAGAGCGATGTTACGGTTTGTTACTG |
| 141(+1) | D L M S H S G T S L T Q |
| | S V A R A M L R F V T |
| 491 | TGACAGCTGAAGCTTTACGTTTTCGGCAAATACAGAGGGGATTTCGTACAACACTGGATGATCTCAGTGG |
| 164(+1) | V T A E A L R F R Q I Q |
| | R G F R T T L D D L S G |
| 561 | GCGTTCTTATGTAATGACTGCTGAAGATGTTGATCTTACATTGAACTGGGGAAGGTTGAGTAGCGTCCTG |
| 188(+1) | R S Y V M T A E D V D L |
| | T L N W G R L S S V L |
| 631 | CCTGACTATCATGGACAAGACTCTGTTCGTGTAGGAAGAATTTCTTTTGGAAGCATTAATGCAATTCTGG |
| 211(+1) | P D Y H G Q D S V R V G |
| | R I S F G S I N A I L |
| 701 | GAAGCGTGGCATTAATACTGAATTGTCATCATCATGCATCGCGAGTTGCCAGAATGGCATCTGATGAGTT |
| 234(+1) | G S V A L I L N C H H H |
| | A S R V A R M A S D E F |
| 771 | TCCTTCTATGTGTCCGGCAGATGGAAGAGTCCGTGGGATTACGCACAATAAAATATTGTGGGATTCATCC |
| 258(+1) | P S M C P A D G R V R G |
| | I T H N K I L W D S S |
| 841 | ACTCTGGGGGCAATTCTGATGCGCAGAACTATTAGCAGTTGA |
| 281(+1) | T L G A I L M R R T I S |
| | S * |

By “wild-type Stx2A,” it is meant the mature Stx2 A subunit amino acid sequence 1-297, excluding the 22-amino acid N-terminal signal peptide (“the N-terminal signal sequence of wild-type Stx2”). Thus, by the term “wild-type Stx2A”, or “Stx2A”, it is meant the Stx2 amino acid sequence 1-297 (hereinafter Stx2 (1-297)). The sequences designated SEQ ID NOS:3 and 4 are the DNA and corresponding amino acid sequence of wild-type Stx2A:

| SEQ ID NO: 3 - Stx2A nucleic acid sequence | |
|---|---|
| SEQ ID NO: 4 - Stx2A amino acid sequence | |
| | 1 11 21 |
| | 31 41 51 61 |
| 1 | CGGGAGTTTACGATAGACTTTTCGACCCAACAAAGTTATGTCTCTTCGTTAAATAGTATACGGACAGAGA |
| 1(+1) | R E F T I D F S T Q Q |
| | S Y V S S L N S I R T E |
| 71 | TATCGACCCCTCTTGAACATATATCTCAGGGGACCACATCGGTGTCTGTTATTAACCACACCCCACCGGG |
| 24(+1) | I S T P L E H I S Q G T |
| | T S V S V I N H T P P G |
| 141 | CAGTTATTTTGCTGTGGATATACGAGGGCTTGATGTCTATCAGGCGCGTTTTGACCATCTTCGTCTGATT |
| 48(+1) | S Y F A V D I R G L D V |
| | Y Q A R F D H L R L I |
| 211 | ATTGAGCAAAATAATTTATATGTGGCCGGGTTCGTTAATACGGCAACAAATACTTTCTACCGTTTTTCAG |
| 71(+1) | I E Q N N L Y V A G F V |
| | N T A T N T F Y R F S |
| 281 | ATTTTACACATATATCAGTGCCCGGTGTGACAACGGTTTCCATGACAACGGACAGCAGTTATACCACTCT |
| 94(+1) | D F T H I S V P G V T T |
| | V S M T T D S S Y T T L |
| 351 | GCAACGTGTCGCAGCGCTGGAACGTTCCGGAATGCAAATCAGTCGTCACTCACTGGTTTCATCATATCTG |
| 118(+1) | Q R V A A L E R S G M Q |
| | I S R H S L V S S Y L |
| 421 | GCGTTAATGGAGTTCAGTGGTAATACAATGACCAGAGATGCATCCAGAGCAGTTCTGCGTTTTGTCACTG |
| 141(+1) | A L M E F S G N T M T R |
| | D A S R A V L R F V T |
| 491 | TCACAGCAGAAGCCTTACGCTTCAGGCAGATACAGAGAGAATTTCGTCAGGCACTGTCTGAAACTGCTCC |
| 164(+1) | V T A E A L R F R Q I Q |
| | R E F R Q A L S E T A P |
| 561 | TGTGTATACGATGACGCCGGGAGACGTGGACCTCACTCTGAACTGGGGGCGAATCAGCAATGTGCTTCCG |
| 188(+1) | V Y T M T P G D V D L T |
| | L N W G R I S N V L P |
| 631 | GAGTATCGGGGAGAGGATGGTGTCAGAGTGGGGAGAATATCCTTTAATAATATATCAGCGATACTGGGGA |
| 211(+1) | E Y R G E D G V R V G R |
| | I S F N N I S A I L G |
| 701 | CTGTGGCCGTTATACTGAATTGCCATCATCAGGGGGCGCGTTCTGTTCGCGCCGTGAATGAAGAGAGTCA |
| 234(+1) | T V A V I L N C H H Q G |
| | A R S V R A V N E E S Q |
| 771 | ACCAGAATGTCAGATAACTGGCGACAGGCCTGTTATAAAAATAAACAATACATTATGGGAAAGTAATACA |
| 258(+1) | P E C Q I T G D R P V |
| | I K I N N T L W E S N T |
| 841 | GCTGCAGCGTTTCTGAACAGAAAGTCACAGTTTTTATATACAACGGGTAAATAA |
| 281(+1) | A A A F L N R K S Q F L |
| | Y T T G K * |

As referred to herein, mutants useful in the practice of various aspects of the present invention include non-wild-type Stx1A1 and Stx2A1 subunits, i.e. Stx1 (1-251) and Stx2 (1-247), respectively. The DNA and corresponding amino acid sequences of Stx1 (1-251) and Stx2 (1-247) designated as SEQ ID NOS:5, 6, 7 and 8 are set forth below.

SEQ ID NO: 5 - Stx1A1 nucleic acid sequence
SEQ ID NO: 6 - Stx1A1 amino acid sequence

| | 1 11 21 |
|---|---|
| | 31 41 51 61 |
| 1 | AAGGAATTTACCTTAGACTTCTCGACTGCAAAGACGTATGTAGATTCGCTGAATGTCATTCGCTCTGCAA |
| 1(+1) | K E F T L D F S T A K T |
| | Y V D S L N V I R S A |
| 71 | TAGGTACTCCATTACAGACTATTTCATCAGGAGGTACGTCTTTACTGATGATTGATAGTGGCTCAGGGGA |
| 24(+1) | I G T P L Q T I S S G G |
| | T S L L M I D S G S G D |
| 141 | TAATTTGTTTGCAGTTGATGTCAGAGGGATAGATCCAGAGGAAGGGCGGTTTAATAATCTACGGCTTATT |
| 48(+1) | N L F A V D V R G I D P |
| | E E G R F N N L R L I |
| 211 | GTTGAACGAAATAATTTATATGTGACAGGATTTGTTAACAGGACAAATAATGTTTTTTATCGCTTTGCTG |
| 71(+1) | V E R N N L Y V T G F V |
| | N R T N N V F Y R F A |
| 281 | ATTTTTCACATGTTACCTTTCCAGGTACAACAGCGGTTACATTGTCTGGTGACAGTAGCTATACCACGTT |
| 94(+1) | D F S H V T F P G T T A V |
| | T L S G D S S Y T T L |
| 351 | ACAGCGTGTTGCAGGGATCAGTCGTACGGGGATGCAGATAAATCGCCATTCGTTGACTACTTCTTATCTG |
| 118(+1) | Q R V A G I S R T G M |
| | Q I N R H S L T T S Y L |
| 421 | GATTTAATGTCGCATAGTGGAACCTCACTGACGCAGTCTGTGGCAAGAGCGATGTTACGGTTTGTTACTG |
| 141(+1) | D L M S H S G T S L T Q |
| | S V A R A M L R F V T |
| 491 | TGACAGCTGAAGCTTTACGTTTTCGGCAAATACAGAGGGGATTTCGTACAACACTGGATGATCTCAGTGG |
| 164(+1) | V T A E A L R F R Q I Q |
| | R G F R T T L D D L S G |
| 561 | GCGTTCTTATGTAATGACTGCTGAAGATGTTGATCTTACATTGAACTGGGGAAGGTTGAGTAGCGTCCTG |
| 188(+1) | R S Y V M T A E D V D L |
| | T L N W G R L S S V L |
| 631 | CCTGACTATCATGGACAAGACTCTGTTCGTGTAGGAAGAATTTCTTTTGGAAGCATTAATGCAATTCTGG |
| 211(+1) | P D Y H G Q D S V R V G |
| | R I S F G S I N A I L |
| 701 | GAAGCGTGGCATTAATACTGAATTGTCATCATCATGCATCGCGAGTTGCCAGA |
| 234(+1) | G S V A L I L N C H H H |
| | A S R V A R |
| SEQ ID NO: 7 - Stx2A1 nucleic acid sequence | |
| SEQ ID NO: 8 - Stx2A1 amino acid sequence | |
| | 1 11 21 |
| | 31 41 51 61 |
| 1 | CGGGAGTTTACGATAGACTTTTCGACCCAACAAAGTTATGTCTCTTCGTTAAATAGTATACGGACAGAGA |
| 1(+1) | R E F T I D F S T Q Q |
| | S Y V S S L N S I R T E |
| 71 | TATCGACCCCTCTTGAACATATATCTCAGGGGACCACATCGGTGTCTGTTATTAACCACACCCCACCGGG |
| 24(+1) | I S T P L E H I S Q G T |
| | T S V S V I N H T P P G |
| 141 | CAGTTATTTTGCTGTGGATATACGAGGGCTTGATGTCTATCAGGCGCGTTTTGACCATCTTCGTCTGATT |
| 48(+1) | S Y F A V D I R G L D V |
| | Y Q A R F D H L R L I |
| 211 | ATTGAGCAAAATAATTTATATGTGGCCGGGTTCGTTAATACGGCAACAAATACTTTCTACCGTTTTTCAG |
| 71(+1) | I E Q N N L Y V A G F V |
| | N T A T N T F Y R F S |
| 281 | ATTTTACACATATATCAGTGCCCGGTGTGACAACGGTTTCCATGACAACGGACAGCAGTTATACCACTCT |
| 94(+1) | D F T H I S V P G V T T |
| | V S M T T D S S Y T T L |
| 351 | GCAACGTGTCGCAGCGCTGGAACGTTCCGGAATGCAAATCAGTCGTCACTCACTGGTTTCATCATATCTG |
| 118(+1) | Q R V A A L E R S G M Q |
| | I S R H S L V S S Y L |
| 421 | GCGTTAATGGAGTTCAGTGGTAATACAATGACCAGAGATGCATCCAGAGCAGTTCTGCGTTTTGTCACTG |
| 141(+1) | A L M E F S G N T M T R |
| | D A S R A V L R F V T |
| 491 | TCACAGCAGAAGCCTTACGCTTCAGGCAGATACAGAGAGAATTTCGTCAGGCACTGTCTGAAACTGCTCC |
| 164(+1) | V T A E A L R F R Q I Q |
| | R E F R Q A L S E T A P |
| 561 | TGTGTATACGATGACGCCGGGAGACGTGGACCTCACTCTGAACTGGGGGCGAATCAGCAATGTGCTTCCG |
| 188 (+1) | V Y T M T P G D V D L T |
| | L N W G R I S N V L P |
| 631 | GAGTATCGGGGAGAGGATGGTGTCAGAGTGGGGAGAATATCCTTTAATAATATATCAGCGATACTGGGGA |
| 211 (+1) | E Y R G E D G V R V G R |
| | I S F N N I S A I L G |
| 701 | CTGTGGCCGTTATACTGAATTGCCATCATCAGGGGGCGCGT |
| 234 (+1) | T V A V I L N C H H Q G |
| | A R |
| 771 | ACCAGAATGTCAGATAACTGGCGACAGGCCTGTTATAAAAATAAACAATACATTATGGGAAAGTAATACA |
| 258(+1) | P E C Q I T G D R P V |
| | I K I N N T L W E S N T |
| 841 | GCTGCAGCGTTTCTGAACAGAAAGTCACAGTTTTTATATACAACGGGTAAATAA |
| 281(+1) | A A A F L N R K S Q F L |
| | Y T T G K * |

Generally, Stx1 and Stx2 mutants differ from wild-type Stx1 and Stx2 in terms of one or more amino acid substitutions, deletions or additions. In some embodiments, the Stx1 and Stx2 mutants differ from wild-type, mature Stx1 and Stx2 exclusively or substantially in that they contain one or more (e.g., two or three) amino acid substitutions at any of positions Stx1 or Stx2 respectively. In other embodiments, the mutants are fragments of wild-type Stx1 or Stx2, in that one or more amino acid residues are deleted from the N-terminus and/or C-terminus. In yet other embodiments, the Stx1 or Stx2 mutants or fragments of wild-type Stx1 or Stx2 respectively and which also contain one or more (e.g., two or three) amino acid substitutions at any of positions 1-253 or 1-247 respectively, and/or deletions of certain numbers of C-terminal amino acid residues.

One category of Stx mutants is characterized by, among other possible changes which may be present or not, at least one amino acid substitution in the A1 subunit. In some embodiments, the mutants contain at least two amino, or even three or more substitutions. The amino acid substitutions may be conservative or non-conservative in nature, depending upon whether the change does not result in a mutant that is toxic, as that term is used in the context of the present invention. Conservative acid substitutions refer to the interchangeability of residues having similar side chains. Conservatively substituted amino acids can be grouped according to the chemical properties of their side chains. For example, one grouping of amino acids includes those amino acids have neutral and hydrophobic side chains (A, V, L, I, P, W, F, and M); another grouping is those amino acids having neutral and polar side chains (G, S, T, Y, C, N, and Q); another grouping is those amino acids having basic side chains (K, R, and H); another grouping is those amino acids having acidic side chains (D and E); another grouping is those amino acids having aliphatic side chains (G, A, V, L, and I); another grouping is those amino acids having aliphatic-hydroxyl side chains (S and T); another grouping is those amino acids having amine-containing side chains (N, Q, K, R, and H); another grouping is those amino acids having aromatic side chains (F, Y, and W); and another grouping is those amino acids having sulfur-containing side chains (C and M). Thus, non-conservative amino acid substitutions refer to the substitution of the residue in the wild-type sequence with any other amino acid sequence.

As shown in the Tables contained in the working examples below, representative examples of Stx1 mutants that differ from wild-type A1 subunit in terms of an amino acid substitution include, but are not limited to Stx1 (1-251, G25D), Stx1 (1-251, G25R), Stx1 (1-251, N75A), Stx1 (1-251, Y77A), Stx1 (1-251,G80E), Stx1 (1-251, G8OR), Stx1 (1-251, S96Y), Stx (1-251, A155R), Stx1 (1-251, E167A), Stx1 (1-251, E167K) and Stx1 (1-251, R170A). The abbreviation Stx1 (1-251, G25D) thus refers to a Stx1 mutant containing a non-wild-type A1 subunit wherein the G residue at position 25 has been changed to D. All other nomenclature used herein with respect to description of Stx mutants is consistent in these respects. In describing the mutants in this fashion, it is not meant to exclude additional amino acid residues that may be present, e.g., residues contained in the A subunit that are C-terminal to the A1 subunit.

Embodiments which include two amino substitutions in the Stx1 A1 subunit include, but are not limited to Stx1 (1-251, D58N, G177R), Stx1 (1-251, V78M, N83D), Stx1 (1-251, A166T, A250V), Stx1 (1-251, R119C, R289K), Stx1 (1-251, S134L, A251G), Stx1 (1-251, E167A, R170A) and Stx1 (1-251, E167K, R176K).

Another category of Stx mutants is characterized by, among other possible changes which may be present or not, deletion of C-terminal amino acid residues in the A1 subunit. Representative examples of such mutants having deletions of C-terminal residues in the A1 subunit of Stx1 include Stx1 (1-202), Stx1 (1-203), Stx1 (1-205), Stx1 (1-206), Stx1 (1-207), Stx1 (1-208), Stx1 (1-209), Stx1 (1-210), Stx1 (1-211), Stx1 (1-212), Stx1 (1-213), Stx1 (1-214), Stx1 (1-215), Stx1 (1-216), Stx1 (1-217), Stx1 (1-218), Stx1 (1-219), Stx1 (1-220), Stx1 (1-221), Stx1 (1-222), Stx1 (1-223), Stx1 (1-224), Stx1 (1-225), Stx1 (1-226), Stx1 (1-227), Stx1 (1-228), Stx1 (1-229), Stx1 (1-230), Stx1 (1-231), Stx1 (1-232), Stx1 (1-233), Stx1 (1-234), Stx1 (1-235), Stx1 (1-236), Stx1 (1-237), Stx1 (1-238) and Stx1 (1-239).

Representative examples of Stx2 mutants that differ from wild-type in terms of an amino acid substitution include, but are not limited to Stx2 (1-247, N75A), Stx2 (1-247, Y77A), Stx2 (1-247, E167A), Stx2 (1-247, R170A) and Stx2 (1-247, R170H).

Embodiments which include two amino substitutions in the Stx2 Al subunit include, but are not limited to Stx2 (1-297, E167K, R176K).

Representative examples of such mutants having deletions of C-terminal residues in the Al subunit of Stx2 include deletions of 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192 to 193 C-terminal amino acid residues. Thus, in some embodiments, the Stx2 mutants include non-wild-type Al subunits having the designations Stx2(1-238),Stx2 (1-237), Stx2 (1-236), Stx2 (1-235), Stx2 (1-234), Stx2 (1-233), Stx2 (1-232), Stx2 (1-231), Stx2 (1-230), Stx2 (1-229), Stx2 (1-228), Stx2 (1-227), Stx2 (1-226), Stx2 (1-225), Stx2 (1-224), Stx2 (1-223), Stx2 (1-222), Stx2 (1-221), Stx2 (1-220), Stx2 (1-219), Stx2 (1-218), Stx2 (1-217), Stx2 (1-216), Stx2 (1-215), Stx2 (1-214), Stx2 (1-213), Stx2 (1-212), Stx2 (1-211), Stx2 (1-210), Stx2 (1-209), Stx2 (1-208), Stx2 (1-207), Stx2 (1-206), Stx2 (1-205), Stx2 (1-204), Stx2 (1-203), Stx2 (1-202), Stx2 (1-201), Stx2 (1-200), Stx2 (1-199), Stx2 (1-198), Stx2 (1-197), Stx2 (1-196), Stx2 (1-195), Stx2 (1-194), Stx2 (1-193), Stx2 (1-192), Stx2 (1-191), Stx2 (1-190), Stx2 (1-189), Stx2 (1-188), Stx2 (1-187), Stx2 (1-186), Stx2 (1-185), Stx2 (1-184), Stx2 (1-183), Stx2 (1-182), Stx2 (1-181), Stx2 (1-180), Stx2 (1-179), Stx2 (1-178), Stx2 (1-177), Stx2 (1-176), Stx2 (1-175), Stx2 (1-174), Stx2 (1-173), Stx2 (1-172), Stx2 (1-171), Stx2 (1-170), Stx2 (1-169), Stx2 (1-168), Stx2 (1-167), Stx2 (1-166), Stx2 (1-165), Stx2 (1-164), Stx2 (1-163), Stx2 (1-162), Stx2 (1-161), Stx2 (1-160), Stx2 (1-159), Stx2 (1-158), Stx2 (1-157), Stx2 (1-156), Stx2 (1-155), Stx2 (1-154), Stx2 (1-153), Stx2 (1-152), Stx2 (1-151), Stx2 (1-150), Stx2 (1-149), Stx2 (1-148), Stx2 (1-147), Stx2 (1-146), Stx2 (1-145), Stx2 (1-144), Stx2 (1-143), Stx2 (1-142), Stx2 (1-141), Stx2 (1-140), Stx2 (1-139), Stx2 (1-138), Stx2 (1-137), Stx2 (1-136), Stx2 (1-135), Stx2 (1-134), Stx2 (1-133), Stx2 (1-132), Stx2 (1-131), Stx2 (1-130), Stx2 (1-129), Stx2 (1-128), Stx2 (1-127), Stx2 (1-126), Stx2 (1-125), Stx2 (1-124), Stx2 (1-123), Stx2 (1-122), Stx2 (1-121), Stx2 (1-120), Stx2 (1-119), Stx2 (1-118), Stx2 (1-117), Stx2 (1-116), Stx2 (1-115), Stx2 (1-114), Stx2 (1-113), Stx2 (1-112), Stx2 (1-111), Stx2 (1-110), Stx2 (1-109), Stx2 (1-108), Stx2 (1-107), Stx2 (1-106), Stx2 (1-105), Stx2 (1-104), Stx2 (1-103), Stx2 (1-102), Stx2 (1-101), Stx2 (1-099), Stx2 (1-98), Stx2 (1-97), Stx2 (1-96), Stx2 (1-95), Stx2 (1-94), Stx2 (1-93), Stx2 (1-92), Stx2 (1-91), Stx2 (1-90), Stx2 (1-89), Stx2 (1-88), Stx2 (1-87), Stx2 (1-86), Stx2 (1-85), Stx2 (1-84), Stx2 (1-83), Stx2 (1-82), Stx2 (1-81), Stx2 (1-80), Stx2 (1-79), Stx2 (1-78), Stx2 (1-77), Stx2 (1-76), Stx2 (1-75), Stx2 (1-74), Stx2 (1-73), Stx2 (1-72), Stx2 (1-71), Stx2 (1-70), Stx2 (1-69), Stx2 (1-68), Stx2 (1-67), Stx2 (1-66), Stx2 (1-65), Stx2 (1-64), Stx2 (1-63), Stx2 (1-62), Stx2 (1-61), Stx2 (1-60), Stx2 (1-59) , Stx2 (1-58) , Stx2 (1-57) , Stx2 (1-56) , Stx2 (1-55) and Stx2 (1-54).

Representative example of a Stx2 mutant that contains a non-wild-type Al subunit that differs from wild-type in terms of an amino acid substitution and a C-terminal deletion is Stx2 (1-179, D111N).

Yet other Stx mutants that may be useful in the present invention may be identified in accordance with the working examples.

Mutants of Stx1 may be constructed generally by methods known in the art. Two such methods are random mutations and site-directed mutations, for example. Both methods are described in the working examples.

It is contemplated herein that the methods of the present invention include administration of one or more Stx mutants alone or in combination with other therapeutic agents, including one or more L3 proteins or fragments as described in U.S. Pat. No. 7,235,715 and U.S. Publication 2006/0005271, and which are incorporated herein by reference.

It is further contemplated herein that the present invention also entails the use of L3 proteins alone or in combination with other agents. Full length L3 proteins and mutants thereof, e.g., N-terminal fragments, may be suitable for use in the present invention.

Ribosomal Protein L3 (RPL3 or L3) is a protein that is part of the ribosome complex. It is one of the first proteins to be assembled into the ribosome. It is known that RPL3 participates in formation of the peptidyltransferase center of the ribosome. The N-terminus of the RPL3 has a nonglobular extension deeply buried inside the ribosome.

L3 nucleic acids and resulting polypeptides useful in the present invention may be obtained from a variety of natural sources including yeast, higher plants and animals. The nucleotide sequence and corresponding amino acid sequence of yeast wild-type L3 protein (known as RPL3) are set forth below as Sequence ID No:9:

```
ATGTCTCACAGAAAGTACGAAGCACCACGTCACGGTCATTTAGGTTTCTTGCCAA GAAAG
1 ----------+---------+---------+---------+---------+--------+
60 TACAGAGTGTCTTTCATGCTTCGTGGTGCAGTGCCAGTAAATCCAAAGAACGGTT
CTTTC a M S H R K Y E A P R H G H L G F L P R K -
AGAGCTGCCTCCATCAGAGCTAGAGTTAAGGCTTTTCCAAAGGATGACAGATCC AAGCCA
61 ---------+---------+---------+---------+---------+-------+
120 TCTCGACGGAGGTAGTCTCGATCTCAATTCCGAAAAGGTTTCCTACTGTCTAGGT
TCGGT a R A A S I R A R V K A F P K D D R S K P -
GTTGCTCTAACTTCCTTCTTGGGTTACAAGGCTGGTATGACCACCATTGTCAGAG ATTTG
121 ----------+---------+---------+---------+---------+------+
180 CAACGAGATTGAAGGAAGAACCCAATGTTCCGACCATACTGGTGGTAACAGTCT
CTAAAC a V A L T S F L G Y K A G M T T I V R D L -
GACAGACCAGGTTCTAAGTTCCACAAGCGTGAAGTTGTCGAAGCTGTCACCGTTG TTGAC
181 ---------+---------+---------+---------+---------+------+
240 CTGTCTGGTCCAAGATTCAAGGTGTTCGCACTTCAACAGCTTCGACAGTGGCAAC
AACTG a D R P G S K F H K R E V V E A V T V V D -
ACTCCACCAGTTGTCGTTGTTGGTGTTGTCGGTTACGTCGAAACCCCAAGAGGTT TGAGA
241 ----------+---------+---------+---------+---------+------+
300 TGAGGTGGTCAACAGCAACAACCACAACAGCCAATGCAGCTTTGGGGTTCTCCA
AACTCT a T P P V V V V G V V G Y V E T P R G L R -
TCTTTGACCACCGTCTGGGCTGAACATTTGTCTGACGAAGTCAAGAGAAGATTCT ACAAG
301 ----------+---------+---------+---------+---------+------+
360 AGAAACTGGTGGCAGACCCGACTTGTAAACAGACTGCTTCAGTTCTCTTCTAAGA
TGTTC a S L T T V W A E H L S D E V K R R F Y K -
AACTGGTACAAGTCTAAGAAGAAGGCTTTCACCAAATACTCTGCCAAGTACGCTC AAGAT
361 ----------+---------+---------+---------+---------+------+
420 TTGACCATGTTCAGATTCTTCTTCCGAAAGTGGTTTATGAGACGGTTCATGCGAG
TTCTA a N W Y K S K K K A F T K Y S A K Y A Q D -
GGTGCTGGTATTGAAAGAGAATTGGCTAGAATCAAGAAGTACGCTTCCGTCGTC AGAGTT
421 ----------+---------+---------+---------+--------+-------+
480 CCACGACCATAACTTTCTCTTAACCGATCTTAGTTCTTCATGCGAAGGCAGCAGT
CTCAA a G A G I E R E L A R I K K Y A S V V R V -
TTGGTCCACACTCAAATCAGAAAGACTCCATTGGCTCAAAAGAAGGCTCATTTGG CTCAA
481 ----------+---------+---------+---------+--------+-------+
540 AACCAGGTGTGAGTTTAGTCTTTCTGAGGTAACCGAGTTTTCTTCCGAGTAAACC
GACTT a L V H T Q I R K T P L A Q K K A H L A E -
ATCCAATTGAACGGTGGTTCCATCTCTGAAAAGGTTGACTGGGCTCGTGAACATT TCGAA
541 ----------+---------+---------+---------+--------+-------+
600 TAGGTTAACTTGCCACCAAGGTAGAGACTTTTCCAACTGACCCGAGCACTTGTAA
AGCTT a I Q L N G G S I S E K V D W A R E H F E -
AAGACTGTTGCTGTCGACAGCGTTTTTGAACAAAACGAAATGATTGACGCTATTG CTGTC
601 ----------+---------+---------+---------+--------+-------+
660 TTCTGACAACGACAGCTGTCGCAAAAACTTGTTTTGCTTTACTAACTGCGATAAC
GACAG a K T V A V D S V F E Q N E M I D A I A V -
ACCAAGGGTCACGGTTTCGAAGGTGTTACCCACAGATGGGGTACTAAGAAATTG CCAAGA
661 ----------+---------+---------+---------+--------+-------+
720 TGGTTCCCAGTGCCAAAGCTTCCACAATGGGTGTCTACCCCATGATTCTTTAACG
GTTCT a T K G H G F E G V T H R W G T K K L P R -
AAGACTCACAGAGGTCTAAGAAAGGTTGCTTGTATTGGTGCTTGGCATCCAGCCC ACGTT
721 ----------+---------+---------+---------+--------+-------+
780 TTCTGAGTGTCTCCAGATTCTTTCCAACGAACATAACCACGAACGGTAGGTCGGG
TGCAA a K T H R G L R K V A C I G A W H P A H V -
ATGTGGAGTGTTGCCAGAGCTGGTCAAAGAGGTTACCATTCCAGAACCTCCATTA ACCAC
781 ----------+---------+---------+---------+--------+-------+
840 TACACCTCACAACGGTCTCGACCAGTTTCTCCAATGGTAAGGTCTTGGAGGTAAT
TGGTG a M W S V A R A G Q R G Y H S R T S I N H -
AAGATTTACAGAGTCGGTAAGGGTGATGATGAAGCTAACGGTGCTACCAGCTTC GACAGA
841 ----------+---------+---------+---------+--------+-------+
                                                                                                                      900
TTCTAAATGTCTCAGCCATTCCCACTACTACTTCGATTGCCACGATGGTCGAAGCT GTCT
a K I Y R V G K G D D E A N G A T S F D R -
ACCAAGAAGACTATTACCCCAATGGGTGGTTTCGTCCACTACGGTGAAATTAAGA ACGAC
901 ----------+---------+---------+---------+--------+-------+
960 TGGTTCTTCTGATAATGGGGTTACCCACCAAAGCAGGTGATGCCACTTTAATTCT
TGCTG a T K K T I T P M G G F V H Y G E I K N D -
TTCATCATGGTTAAAGGTTGTATCCCAGGTAACAGAAAGAGAATTGTTACTTTGA GAAAG
961 ----------+---------+---------+---------+--------+-------+
                                                                                                                    1020
AAGTAGTACCAATTTCCAACATAGGGTCCATTGTCTTTCTCTTAACAATGAAACT CTTTC
a F I M V K G C I P G N R K R I V T L R K -
TCTTTGTACACCAACACTTCTAGAAAGGCTTTGGAAGAAGTCAGCTTGAAGTGGA TTGAC
1021 ---------+---------+---------+---------+--------+-------+
                                                                                                                    1080
AGAAACATGTGGTTGTGAAGATCTTTCCGAAACCTTCTTCAGTCGAACTTCACCT AACTG
a S L Y T N T S R K A L E E V S L K W I D -
ACTGCTTCTAAGTTCGGTAAGGGTAGATTCCAAACCCCAGCTGAAAAGCATGCTT TCATG
1081 ---------+---------+---------+---------+--------+-------+
                                                                                                                    1140
TGACGAAGATTCAAGCCATTCCCATCTAAGGTTTGGGGTCGACTTTTCGTACGAA AGTAC
a T A S K F G K G R F Q T P A E K H A F M -
GGTACTTTGAAGAAGGACTTGTAA 1141 -----------+----------+---- 1164
CCATGAAACTTCTTCCTGAACATT a G T L K K D L * -
```

In addition to full length L3 proteins, N-terminal fragments may also be useful. The L3Δ99 peptide as referred to herein includes DNA sequences that encode a polypeptide having at least the first 21 N-terminal amino acid residues and as many as about the first 99 N-terminal amino acid residues of a full-length eukaryotic L3 protein (hereinafter “L3 N-terminal polypeptides”, or “L3 N-terminal polypeptide fragments,” or an analog of the L3 polypeptide. Eucaryotic L3 proteins include, but are not limited to human, yeast, bovine, mice, rat and higher plant (e.g., rice wheat, barley, and tobacco) and Arabidopsis L3 proteins. An alignment of the amino acid sequences of full-length L3 proteins from Arabidopsis (i.e., AtRPL3A and AthRPL3B), Nicotiana tabacum (i.e., NtRPL3-8d and NtRPL3-10d), yeast (i.e., YRPL3), and rice (i.e., HvRPL3) various L3 proteins, and their first 100 amino acid residues, are illustrated in FIG. 13.

L3Δ99 peptides may include the first 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 and 99 N-terminal amino acid residues of a eukaryotic L3 protein. These polypeptides are referred to herein as L3(1-21), L3(1-22), L3(1-23), L3(1-24), L3(1-25), L3(1-26), L3(1-27), L3(1-28), L3(1-29), L3(1-30), L3(1-31), L3(1-32), L3(1-33), L3(1-34), L3(1-35), L3(1-36), L3(1-37), L3(1-38), L3(1-39), L3(1-40), L3(1-41), L3(1-42), L3(1-43), L3(1-44), L3(1-45), L3(1-46), L3(1-47), L3(1-48), L3(1-49), L3(1-50), L3(1-51), L3(1-52), L3(1-53), L3(1-54), L3(1-55), L3(1-56), L3(1-57), L3(1-58), L3(1-59), L3(1-60), L3(1-61), L3(1-62), L3(1-63), L3(1-64), L3(1-65), L3(1-66), L3(1-67), L3(1-68), L3(1-69), L3(1-70), L3(1-71), L3(1-72), L3(1-73), L3(1-74), L3(1-75), L3(1-76), L3(1-77), L3(1-78), L3(1-79), L3(1-80), L3(1-81), L3(1-82), L3(1-83), L3(1-84), L3(1-85), L3(1-86), L3(1-87), L3(1-88), L3(1-89), L3(1-90), L3(1-91), L3(1-92), L3(1-93), L3(1-94), L3(1-95), L3(1-96), L3(1-97), L3(1-98) and L3(1-99), respectively. L3(1-99) is also referred to herein, as “L3Δ1-99” or L3Δ99.” By way of specific example, L3Δ99 in yeast has an amino acid (and corresponding nucleotide) sequence as set forth below (Sequence ID No:10 and Sequence ID No:11, respectively):

Yeast L3(1-99):
+1
MSHRKYEAPRHGHLGFLPRKRAASIRARVKAFPKDDRSKPVALTSFLGY
KAGMTTIVRDLDRPGSKFHKREVVEAVTVVDTPPVVVVGVVGYVETPRG
L + 99)
Yeast L3 (1-99) nucleotide
+1
ATGTCTCACAGAAAGTACGAAGCACCACGTCACGGTCATTTAGGTTTCT
TGCCAAGAAAG
AGAGCTGCCTCCATCAGAGCTAGAGTTAAGGCTTTTCCAAAGGATGACA
GATCCAAGCCA
GTTGCTCTAACTTCCTTCTTGGGTTACAAGGCTGGTATGACCACCATTG
TCAGAGATTTG
GACAGACCAGGTTCTAAGTTCCACAAGCGTGAAGTTGTCGAAGCTGTCA
CCGTTGTTGAC
ACTCCACCAGTTGTCGTTGTTGGTGTTGTCGGTTACGTCGAAACCCCAA
GAGGTTTGA +298.

Thus, the amino acid sequences corresponding to yeast L3(1-21) to L3(1-99) may be easily ascertained, as follows:

L3(1-21) MSHRKYEAPRHGHLGFLPRKR;
L(1-22) MSHRKYEAPRHGHLGFLPRKRA;
L3(1-23) MSHRKYEAPRHGHLGFLPRKRAA;
L3(1-24 MSHRKYEAPRHGHLGFLPRKRAAS;
L3(1-25) MSHRKYEAPRHGHLGFLPRKRAASI, etc.
(Sequence Nos:12-16, respectively).

L3 proteins are generally conserved and contain a high level of sequence similarity. However, within the first 99 amino acids there may be some differences. By way of example, those difference may occur at positions 6 (F or Y), 8 (H or A), 11 (H or T) , 13 (S or H), 23 (N, S or A) , 24 (R or S), 25 (H or I), 27 (G or A), 28 (K or R), 29 (V or C), 31 (A or S), 37(Q, P, T, R or K), 38 (T, N, or S), 41 (C or V), 42 (K, R, A, or H), 43 (F or L), 45 (A or S), 47 (M or L), 55 (H or T), 60 (V or L), 61 (E or D), 62 (K or R), 67, (L, F or M), 70 (K or R), 72 (T or V), 73 (C or V), 75 (A or L), 78 (I or V), 79 (I or V), 80 (E or D), 83 (A or P), 84 (M, V or I), 85 (V or I), 86 (V or I), 91 (A or G) and 94 (K or E). Yet other L3Δ99 polypeptides may be based on amino acid sequences of L3 proteins not specifically disclosed herein in accordance by resort to the literature or standard techniques (e.g., probing genomic or cDNA libraries with probes corresponding to conserved regions of L3 proteins.

Depending on the nature of the restriction enzyme and the vector, use of L3(1-99) will result in expression of L3 (1-100). This would occur, for instance, when L3 DNA starting material is produced by treating yeast L3 DNA with BglII, inserting the DNA encoding L3(1-99) into a vector with a BamHI or BglII site, and then transforming a cell with the vector. In this case, an “R” codon would be added. Since native yeast L3 contains an R at residue 100, the corresponding expression product would be L3 (1-100). Thus, L3Δ99 polypeptides include L3 (1-100). L3(1-100) is also referred to herein, as “L3Δ100” or L3Δ1-100.”

Any of the peptides disclosed herein may be further derivatized in terms of amino acid alterations or modifications, substitutions, insertions or deletions, and preferably in terms of one or more conservative or non-conservative amino acid substitutions. It is well understood by the skilled artisan that there is a limit to the number of changes that may be made within a portion of the molecule and still result in a molecule with an acceptable level of equivalent biological activity of function. There are several general guidelines to consider in determining whether a given change in an amino acid sequence will result in an unacceptable change in the desired activity. First, tolerance to change increases with the length of the peptide or protein. It is also well understood that where certain residues are shown to be particularly important to the biological or structural properties of a polyamino acid, such residues may not generally be exchanged. Amino acid substitutions are generally based on the relative similarity of the various types of amino acid side-chains, for example, their hydrophobicity, hydrophilicity, charge, size, and the like. For example, the nonpolar (hydrophobic) amino acids include alanine, leucine, isoleucine, valine, proline, phenylalanine, tryptophan and methionine. Amino acids containing aromatic ring structures are phenylalanine, tryptophan, and tyrosine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid.

Therefore, based upon these considerations, arginine, lysine and histidine; alanine, glycine and serine; and phenylalanine, tryptophan and tyrosine; are defined herein as biologically functional equivalents. To effect more quantitative changes, the hydropathic index of amino acids may be considered. Each amino acid has been assigned a hydropathic index on the basis of its hydrophobicity and charge characteristics, which are as follows: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). The importance of the hydropathic amino acid index in conferring interactive biological function on a protein, and correspondingly a polyamino acid, is generally understood in the art. It is known that certain amino acids may be substituted for other amino acids having a similar hydropathic index or score and still retain a similar biological activity. In making changes based upon the hydropathic index, the substitution of amino acids whose hydropathic indices are within 2 is preferred, those which are within approximately 1 are particularly preferred, and those within approximately 0.5 are even more particularly preferred.

It is also understood in the art that the substitution of like amino acids can be made effectively on the basis of hydrophilicity. As disclosed in U.S. Pat. No. 4,554,101, the following hydrophilicity values have been assigned to amino acid residues: arginine (+3.0); lysine (+3.0); aspartate (+3.0±1); glutamate (+3.0±I); serine 5 (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In making changes based upon similar hydrophilicity values, the substitution of amino acids whose hydrophilicity values are within ±2 is preferred, those which are within ±1 are particularly preferred, and those within ±0.5 are even more particularly preferred. In some embodiments, analogs of the polypeptides contain amino acid substitutions in the positions where variability exists.

The Stx1 and Stx2 mutants and the L3 proteins of the present invention (collectively “the active peptides”) may be made by any of a number of techniques of protein chemistry or molecular biology familiar to one of skill in the art and include synthetic and semi-synthetic chemical synthesis as well as recombinant methods.

The active peptides may be produced using chemical methods in whole or in part and using classical or nonclassical amino acids or chemical amino acid analogs as appropriate. Techniques include solid phase chemistry (Merrifield, J. Am. Chem. Soc., 85:2149, 1964; Houghten, Proc. Natl. Acal. Sci. USA 82:5132, 1985) and equipment for such automated synthesis of polypeptides is commercially available (e.g., Perkin Elmer Biosystems, Inc., Foster City, Calif.). Synthesized peptides can be purified using conventional methods such as high performance liquid chromatography. The composition of the synthetic fusion polypeptides may be confirmed by amino acid analysis or sequencing using techniques familiar to one of skill in the art. Further treatment of a synthesized protein under oxidizing conditions may also be utilized to obtain the proper native conformation. See, e.g. Kelley, R. F. & Winkler, M. E. in Genetic Engineering Principles and Methods, Setlow, J. K., ed., Plenum Press, N.Y., vol. 12, pp 1-19, 1990; Stewart, J. M. & Young, J. D. Solid Phase Peptide Synthesis Pierce Chemical Co., Rockford, Ill.,1984).

The active peptides disclosed herein may also be made by recombinant techniques involving gene synthesis, cloning and expression methodologies. These techniques are well known and are explained in, for example, Current Protocols in Molecular Biology, Volumes I, II, and III, 1997 (F. M. Ausubel ed.); Sambrook et al., 1989, Molecular Cloning: A Laboratory Manual, Second Edition, Cold Spring Harbor Laboratory Press, Cold Spring Harbor, N.Y.; DNA Cloning: A Practical Approach, Volumes I and II, 1985 (D. N. Glover ed.); A Practical Guide to Molecular Cloning; the series, Methods in Enzymology (Academic Press, Inc.); Gene Transfer Vectors for Mammalian Cells, 1987 (J. H. Miller and M. P. Calos eds., Cold Spring Harbor Laboratory); and Methods in Enzymology Vol. 154 and Vol. 155 (Wu and Grossman, and Wu, eds., respectively).

Briefly, the active peptides of the present invention may be made recombinantly by isolating or synthesizing nucleic acid sequences encoding any of the amino acid sequences described herein by conventional cloning or chemical synthesis methods. For example, DNA fragments coding for the different active peptides may be ligated together in-frame in accordance with conventional techniques or synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence. The recombinant nucleic acids can further comprise other nucleotide sequences such as sequences that encode affinity tags to facilitate protein purification protocol.

The nucleic acid sequence encoding a Stx1 or Stx2 mutant of the present invention may be ligated into a suitable expression vector capable of expressing the nucleic acid sequence in a suitable host, followed by transforming the host with the expression vector into which the nucleic acid sequence has been ligated, culturing the host under conditions suitable for expression of the nucleic acid sequence, whereby the protein encoded by the selected nucleic acid sequence is expressed by the host and purifying the protein produced. In this process, the ligating step may further contemplate ligating the nucleic acid into a suitable expression vector such that the nucleic acid is operably linked to a suitable secretory signal, whereby the amino acid sequence is secreted by the host. Suitable secretory signals for use with the present invention include but are not limited to, the mouse IgG kappa light chain signal sequence (Ho et. al. PNAS (2006) 103(25): 9637-9642). The use of mammalian, prokaryotic, yeast, plant or transgenic expression systems to create the Stx1 and Stx2 mutants disclosed herein is contemplated herein and such techniques are familiar to one of skill in the art.

As described above, a nucleic acid sequence encoding an active peptide described herein may be inserted into an appropriate plasmid or expression vector that may be used to transform a host cell. In general, plasmid vectors containing replication and control sequences that are derived from species compatible with the host cell are used in connection with those hosts. The vector ordinarily carries a replication site, as well as sequences which encode proteins that are capable of providing phenotypic selection in transformed cells. For example, E. coli may be transformed using pBR322, a plasmid derived from an E. coli species (Mandel, M. et al., J. Mol. Biol. 53:154,1970). Plasmid pBR322 contains genes for ampicillin and tetracycline resistance, and thus provides easy means for selection. Other vectors include different features such as different promoters, which are often important in expression. The vectors used for mammalian expression often contain the constitutive CMV promoter that leads to high recombinant protein expression. These vectors also contain selection sequence that are used for the generation of stable expressing cell lines.

Host cells may be prokaryotic or eukaryotic. Prokaryotes are preferred for cloning and expressing DNA sequences to produce parent polypeptides, segment substituted polypeptides, residue-substituted polypeptides and polypeptide variants. Such prokaryotic cells familiar to one skilled in the art include, but are not limited to, E. coli, B subtillus, and P. aeruginosa cell strains. In addition to prokaryotes, eukaryotic organisms, such as yeast cultures, or cells derived from multicellular organisms may be used. Vertebrate cells may also be used as useful host cell lines. Useful cells and cell lines are familiar to one of skill in the art and include, but are not limited to, HEK293 cells, HeLa cells, Chinese Hamster Ovary (CHO) cell lines, W138, 293, BHK, COS-7 and MDCK cell lines.

Another aspect of the present invention relates to isolated or purified polynucleotides that encode the Stx1 and Stx2 mutants. As discussed above, the polynucleotides of the invention which encode a Stx1 or Stx2 mutant may be used to generate recombinant nucleic acid molecules that direct the expression of the Stx1 or Stx2 mutant in appropriate host cells. The fusion polypeptide products encoded by such polynucleotides may be altered by molecular manipulation of the coding sequence.

Due to the inherent degeneracy of the genetic code, other DNA sequences which encode substantially the same or a functionally equivalent amino acid sequence, may be used in the practice of the invention for the expression of the fusion polypeptides. Such DNA sequences include those which are capable of hybridizing to the coding sequences or their complements disclosed herein under low, moderate or high stringency conditions described herein.

Altered nucleotide sequences which may be used in accordance with the invention include deletions, additions or substitutions of different nucleotide residues resulting in a sequence that encodes the same or a functionally equivalent gene product. The gene product itself may contain deletions, additions or substitutions of amino acid residues, which result in a silent change.

The nucleotide sequences of the invention may be engineered in order to alter the Stx1 or Stx2 mutant coding sequence for a variety of ways, including but not limited to, alterations which modify processing and expression of the gene product. For example, mutations may be introduced using techniques which are well known in the art, e.g., to insert or delete restriction sites, to alter glycosylation patterns, phosphorylation, to create and/or destroy translation, initiation, and/or termination sequences, or to create variations in coding regions, to facilitate further in vitro modification, etc. One of skill will recognize many ways of generating alterations in a given nucleic acid construct. Such well-known methods include, e.g., site-directed mutagenesis, PCR amplification using degenerate oligonucleotides, exposure of cells containing the nucleic acid to chemical mutagenic agents or radiation, chemical synthesis of a desired oligonucleotide (e.g., in conjunction with ligation and/or cloning to generate large nucleic acids) and other well-known techniques.

Purified Stx1 and Stx2 mutants may be prepared by culturing suitable host/vector systems to express the recombinant translation products of the DNAs of the present invention, which are then purified from culture media or cell extracts in accordance with standard techniques in the field of protein purification. For example, supernatants from systems which secrete recombinant polypeptide into culture media may be first concentrated using a commercially available protein concentration filter, such as, e.g., an Amicon or Millipore Pellicon ultrafiltration unit. Following the concentration step, the concentrate may be applied to a suitable purification matrix. Affinity chromatography or reverse-phase high performance liquid chromatography (RP-HPLC) may also be used to purify the Stx1 and Stx2 mutants of the present invention.

The active peptides of the present invention may or may not be glycosylated. For example, Stx1 and Stx2 mutants expressed in yeast or mammalian expression systems may be similar to, or slightly different in molecular weight and glycosylation pattern from the native molecules, depending upon the expression system; expression of DNA encoding polypeptides in bacteria such as E. coli provides non-glycosylated molecules.

Stx1 and Stx2 mutants described herein may be administered in the form of a vaccine to elicit an immune response. Such methods entail administering the Stx1 and/or Stx2 mutants which without intending to be bound by theory, are believed to act as an active immunogenic agent to induce a beneficial immune response including host production of antibodies against Stx1 and/or Stx2 in a patient in need thereof e.g., humans at risk of or suspected to have had exposure to Stx-producing microorganisms. Such methods may be carried out by conventional modes of administration known to those skilled in the art.

In this regard, it is also contemplated that the mutant peptides described herein may be used in the generation of antibodies against Shiga-like toxin for use in passive immunization. For example, a mutant Stx1 or Stx2 peptide linked to a carrier can be administered to a laboratory animal in the production of monoclonal antibodies to Shiga-like toxin. The antibodies may subsequently be administered to a patient in need thereof.

The L3 protein may be administered alone or in combination with the Stx1 or Stx2 mutant. Typically, however, after a confirmatory diagnosis, and/or if a prior administration of the Stx1 or Stx2 mutant was ineffective to thwart the infection.

The active peptides of the invention are typically administered in the form of a pharmaceutical composition comprising the active peptide and one or more other pharmaceutically acceptable (e.g., inert) components. See Remington's Pharmaceutical Science (15th ed., Mack Publishing Company, Easton, Pa., 1980). The preferred form depends on the intended mode of administration and therapeutic application. The compositions typically include, depending on the formulation desired, pharmaceutically-acceptable, non-toxic carriers or diluents, which are defined as vehicles commonly used to formulate pharmaceutical compositions for human administration. The diluent is selected so as not to affect the biological activity of the combination. Examples of such diluents are distilled water, physiological phosphate-buffered saline, Ringer's solutions, dextrose solution, and Hank's solution. The pharmaceutical compositions may also include adjuvants, or nontoxic, nontherapeutic, nonimmunogenic stabilizers and the like. However, some reagents suitable for administration to animals, such as Complete Freund's adjuvant are not typically included in compositions for human use.

Thus, pharmaceutical compositions for use in accordance with the present invention may be formulated in a conventional manner using one or more physiologically acceptable carriers or excipients for administration by various means, for example, by inhalation or insufflation (either through the mouth or the nose), topical or parenteral administration.

The active peptides may be administered by inhalation or insufflation (either through the mouth or the nose). As such, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

In a particular embodiment, the pharmaceutical compositions of the present invention may be formulated for parenteral administration by injection, e.g., by bolus injection or continuous infusion. In addition, as contemplated herein, the active peptides or pharmaceutical compositions of the present invention may be suitable for self-injection by a patient in need thereof. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Alternatively, the active peptides may be in powder form for constitution with a suitable vehicle, e.g., sterile pyrogen-free water, before use. For example, lyophilized protein compositions may be inhaled or reconstituted then injected in a suitable vehicle.

In addition to the formulations described previously, which may exhibit pharmacokinetics similar to a slow release formulation, the compounds may also be formulated as an actual depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

Pharmaceutical compositions suitable for use in the invention include compositions wherein the active peptide(s) are contained in an effective amount to achieve the intended purpose, e.g., treat or ameliorate toxic effects of shiga or shiga-like toxin. The determination of an effective dose is well within the capability of those skilled in the art. For example, for any compound, the therapeutically effective dose can be estimated initially either in cell culture assays, e.g., of neoplastic cells, or in animal models, usually mice, rabbits, dogs, or pigs. The animal model may also be used to determine the appropriate concentration range and route of administration. A dose may be formulated in animal models to achieve a circulating plasma concentration range that includes the IC₅₀ (i.e., the concentration of the test compound that achieves a half-maximal inhibition of symptoms). Such information can then be used to determine useful doses and routes for administration in humans.

A therapeutically effective dose or “effective amount” refers to that amount of active peptide that is nontoxic but sufficient to provide the desired therapeutic effect, e.g., treat or ameliorate toxic effects of shiga or shiga-like toxin. Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between toxic and therapeutic effects is the therapeutic index, and it can be expressed as the ratio, LD50/ED50. Pharmaceutical compositions that exhibit large therapeutic indices are preferred. The data obtained from cell culture assays and animal studies is used in formulating a range of dosage for human use. The dosage contained in such compositions is preferably within a range of circulating concentrations that include the ED50 with little or no toxicity. The dosage varies within this range depending upon the dosage form employed, sensitivity of the patient, and the route of administration.

The exact dosage will be determined by the practitioner, in light of factors related to the subject that requires treatment. Dosage and administration are adjusted to provide sufficient levels of the active peptides or to maintain the desired effect. Factors that may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

Normal dosage amounts on a daily basis may vary from 0.1 to 100,000 micrograms, 1 to 50 micrograms protein per patient, 1 to 100 micrograms protein per patient, even up to a total dose of about 1 g, depending upon the route of administration. Guidance as to particular dosages and methods of delivery is provided in the literature and generally available to practitioners in the art.

The present invention further provides kits for use with any of the above methods. Such kits typically comprise two or more components necessary for performing a method described herein. Components may be compounds, reagents, containers and/or equipment. For example, one container within a kit may contain a pharmaceutical composition comprising a Stx1 and/or Stx2 mutant of the present invention. One or more additional containers may enclose elements, such as reagents or buffers, a pharmaceutical composition containing an L3 protein, or equipment to be used in a method to administer the pharmaceutical composition.

It is also contemplated herein that the active peptides of the present invention may be administered alone or in combination with other compounds or substances that may be used to treat any of the pathological conditions described herein.

The invention will be further described by reference to the following detailed examples. These examples are provided for purposes of illustration only and are not intended to be limiting as to the scope of the invention described herein, unless otherwise specified.

### Example 1

### Random Mutations of Stx1

The full-length cDNA corresponding to the A subunit of Stx1 (Stx1A) including the N-terminal 22-residue signal peptide and 293-residue mature Stx1 was cloned into pGEMT-easy (Promega) vector by PCR using total DNA isolated from E. coli O157:H7. The Stx1A cDNA was then subcloned into pYES2.1/V5-His-Topo yeast expression vector (Invitrogen) downstream of the GAL1 promoter, resulting in NT890 with 3′ V5- and His-tags. NT890 plasmid was mutagenized by hydroxylamine and transformed into yeast. Yeast cells were grown on SD-Ura medium supplemented with glucose. A totally of 111 yeast colonies were singly picked and replica plated on SD-Ura plates containing galactose. The initial screening found 70 (63%) clones growing well on galactose-containing medium. Twenty-eight (28) clones (25%) could grow partially on galactose medium. Plasmids were isolated from some yeast clones and transformed into E. coli DH5α and their nucleotide sequences were determined. Out of these clones, 16 clones were found to contain single point mutations throughout the Stx1 genome (Table 1, Group I). It was also found that 12 clones contained stop codon mutations (some are listed in Table 1, Group II) and 6 clones contained more than one mutation without any stop codons (Group III) throughout the Stx1A genome. Some mutants occurred more than once.

TABLE 1
Stx1A mutants (Y = yes; N = no;
s.d. = site-directed mutants)

| | | | Depurination | Doubling |
|---|---|---|---|---|
| Stx1A mutants | AA change | Cytotoxicity | (% wt) | (hr) |
| | wild type (wt) | Y | 100 | 13.3 |
| | Group I | | | |
| NT2004 | R21H | Y | 40 | 12.9 |
| NT2025 | G25D | N | 100 | 7.3 |
| NT2026 | G25R | N | 100 | 5.7 |
| NT2017 | R63W | Y | 100 | 14.3 |
| NT2034 (s.d.) | N75A | N | 100 | 6.3 |
| NT2014 (s.d.) | Y77A | N | 100 | 6 |
| NT2031 | G80E | N | 79 | 10.1 |
| NT2022 | G80R | N | 100 | 4.3 |
| NT2021 | S96Y | N | 100 | 8.7 |
| NT2044 | S112R | Y | 100 | 14.7 |
| NT2029 | R119C | Y | 10 | 13.2 |
| NT2001 | T137I | Y | 100 | 15.2 |
| NT2015 | A155R | N | 100 | 6.5 |
| NT998 (s.d.) | E167A | N | 50 | 6.5 |
| NT2028 | E167K | N | 19 | 7.8 |
| NT2041 (s.d) | R170A | N | 80 | 10.3 |
| NT2036 | R170C | Y | 100 | 18 |
| NT2030 | R172Q | Y | 100 | 13.1 |
| NT2024 | R179H | Y | 100 | 14.7 |
| NT2007 | G234E | Y | 100 | 19.2 |
| | Group II | | | |
| NT2010 | W203* | N | 0 | 9.6 |
| NT2027 | Q216* | N | 0 | 4.9 |
| NT2038 | G227* | N | 88 | 9.4 |
| NT2032 (s.d.) | V236* | N | 73 | 12.6 |
| NT2049 (s.d.) | A237* | N | | |
| NT2050 (s.d.) | L238* | N | | |
| NT2051 (s.d.) | I239* | N | | |
| NT2040 (s.d.) | L240* | N | 83 | 8.8 |
| NT2042 (s.d.) | N241* | Y | | 12.3 |
| NT2033 (s.d.) | C242* | Y | 49 | 14.7 |
| | Group III | | | |
| NT2006 | D58N/G177R | N | 51 | 8.7 |
| NT2037 | V78M/N83D | N | 68 | 11.8 |
| NT2002 | A166T/A250V | N | 100 | 8.9 |
| NT2008 | R119C/R156K | N | 100 | 4.3 |
| NT2023 | R119C/R289K | N | 100 | 6.5 |
| NT2035 | S134L/A284G | N | 100 | 12.7 |
| NT2039 (s.d.) | E167A/R170A | N | 80 | 5.8 |
| NT2048 (s.d.) | E167K/R176K | N | 0 | |

### Example 2

### Stx1 Mutants not Toxic to Yeast Cells

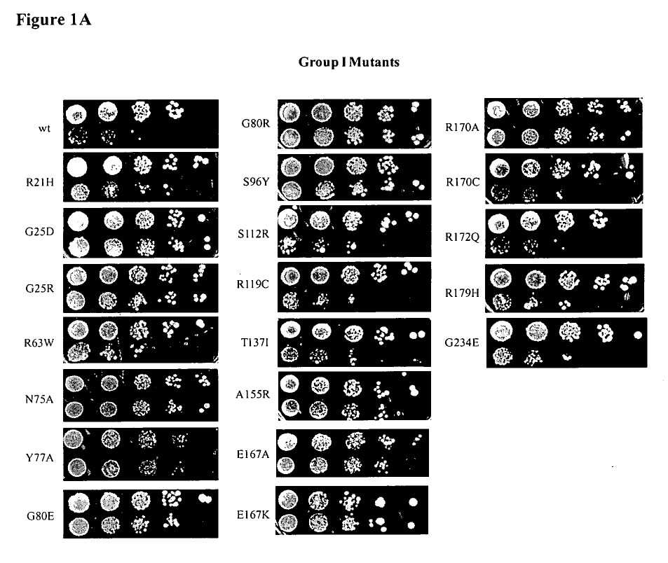

The growth inhibition of yeast cells by Stx1A point mutants was determined by the viability assay. Yeast cells expressing wt Stx1A or the mutants were plated on SD-Ura/glucose medium after induction in galactose for 0 and 10 hours. Upon induction, wt Stx1A reduced the viability of yeast cells by 3 logs at 10 h (FIG. 1). Viability assay showed that some non-stop codon point mutants, G25D, G25R, N75A, Y77A, G80E, G8OR, S96Y, A155R, E167A, E167K and R170A, lost their cytotoxicity significantly because the yeast cells harboring these mutants grew as well as those cells harboring the vector control at 10 h post-induction.

The stop codon mutants, from W203* to L240*, resulting in 3′-truncations have lost their cytotoxicity. The region comprising G227 to R251 is predicted to be the trans-membrane domain of Stx1A by LaPointe et al., A Role for Protease-sensitive Loop Region of Shiga-like Toxin 1 in the Retrotranslocation of Its Al Domain from the Endoplasmic Reticulum Lumen. J. Biol Chem. 280:24 pp. 23310-23318 (2005) have shown that 3′-truncation of Stx1A up to L240* results in the loss of cytotoxicity. Our results here confirmed their findings, as W203* through L240* yeast cells are viable and N241* and C242* cells remained non-viable after 10-hr induction.

All of the double amino acid mutants have lost their cytotoxicity. These include D58N/G177R, V78M/N83D, A166T/A250V, R119C/R156K, R119C/R289K, S134L/A284G, E167A/R170A and E167K/R176K.

### Example 3

### Wild-Type (wt) and Mutant Stx1 Expression and Accumulation in Yeast Membrane

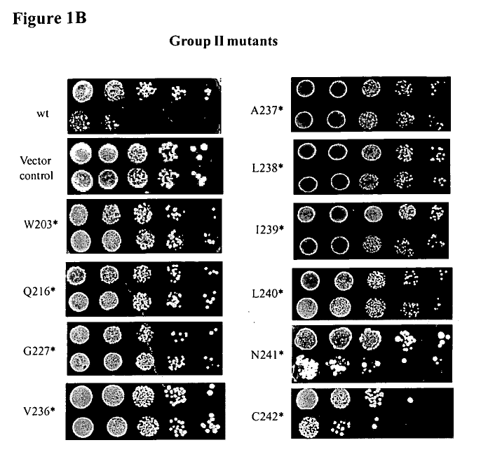

Immunoblot analysis with anti-V5 monoclonal antibody (Invitrogen) was used to examine the protein expression of wt Stx1A and its mutants. Total membrane fractions were separated from the cytosolic fractions of yeast cells harboring wt Stx1A and mutants. Our results have shown that wt Stx1A and mutants are mainly associated with the membranes (FIG. 2). The expected molecular weight of mature Stx1A is approximately 30 kDa if the 22-residue signal peptide is processed. Most of the Stx1A mutants expressed a single or double proteins as the wt Stx1A which was often undetectable.

### Example 4

### Nontoxic Stx1 Mutants Depurinate rRNA

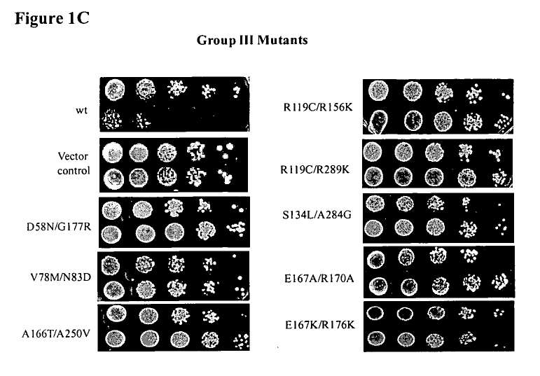

To determine if the reduced cytotoxicity of Stx1A mutants was the result of reduced rRNA depurination, total RNA was isolated from yeast cells harboring wt Stx1A and mutants and subjected to a dual primer extension assay. FIG. 3 shows that some of the Stx1A single point mutants depurinate yeast rRNA as well as the wt Stx1A, namely R63W, S112R, T137I, R170C, R172Q, R179H and G234E. Mutant R21H has a significantly reduced depurination level compared to the wt Stx1A. Nontoxic mutants G80E, E167A, E167K, R170A, D58N/G177R and V78M/N83D have lost their depurination ability by 20 to 81% as compared to wt Stx1A, indicating these amino acids are critical for the depurination capacity of Stx1A. However, some mutants including G25D, G25R, N75A, Y77A, G80R, S96Y, A155R, A166T/A250V, R119C/R156K, R119C/R289K and S134L/A284G, although non-toxic by viability, remained fully capable of depurinating rRNA, indicating the cytotoxicity of Stx1A is not soled resulted from its depurination capability. E167 to R170 are the presumed active site of Stx1A in accordance with other RIPs such as PAP and RTA. The double active site mutations E167A/R170A have been shown to completely abolish the toxicity of Stx1. Our viability assay has also shown that E167A/R170A is non-toxic (FIG. 1), but this mutant has lost its depurination ability by only 20%. It is the other double mutant E167K/R176K that has completely lost its depurinating ability (FIG. 3), indicating R176 might be also critical for Stx1A depurination of rRNA. Mutant R119C is particular in that it was shown to be toxic (FIG. 1) but depurinating at 10% level of the wt Stx1A, implying other mechanisms besides depurination may contribute to the cytotoxicity of Stx1A.

C-terminal stop-codon deletion mutants, W203* and Q216* were not toxic and did not depurinate rRNA (FIG. 3). As mentioned above, deletion up to L240 resulted in mutants that were still toxic, e.g., N241* and C242*. Although G227* downstream to L240* were shown to lose their cytotoxicity completely, their depurination ability was only reduced slightly. These data indicate that C-terminal deletions resulted in the loss of Stx1A toxicity before the loss of depurination ability, further demonstrating that the cytotoxicity of Stx1A is not directly coupled with its depurination. The C-terminus of Stx1A is associated with the ERAD pathway and the ability of Stx1A to retrotranslocate from ER to cytosol may be directly linked with the depurination activity.

### Example 5

### Nontoxic Stx1 Mutants Grow Better Than wt Stx1 in Yeast

The growth of wt Stx1 and Stx1 mutants was monitored after induction with galactose. The yeast cells started with OD600 of around 0.3. The doubling time of yeast cells were recorded as shown in Table 1. Typically the doubling time for wt Stx1 is 13.3 hr. All the nontoxic mutants have much shortened doubling times, which correlated with the reduced cytotoxicity of these mutants.

### Example 6

### Random Mutations of Stx2

The full-length cDNA corresponding to the Stx2A including the N-terminal 22-residue signal peptide and 297-residue mature Stx2A was cloned into pGEMT-easy (Promega) vector by PCR using total DNA isolated from E. coli O157:H7. The Stx2 cDNA was then subcloned into pYES2.1/V5-His-Topo yeast expression vector (Invitrogen) downstream of the GAL1 promoter, resulting in NT901 with the 3′ V5- and His-tags. NT901 plasmid was mutagenized by hydroxylamine and transformed into yeast. Yeast cells were grown on SD-Ura medium supplemented with glucose. Totally 180 yeast colonies were singly picked and replica plated on SD-Ura plates containing galactose. The initial screening found 75 (42%) clones growing well on galactose-containing medium. 64 clones (36%) could grow partially on galactose medium. Plasmids were isolated from some yeast clones and transformed into E. coli DH5α and their nucleotide sequences were determined. Out of these clones, 7 clones were found to contain single mutations throughout the Stx2A genome (Table 2, Group I). It was also found 7 clones contained stop codon mutations (some are listed in Table 2, Group II) and 6 clones contained more than one mutation without any stop codons (Group III) throughout the Stx2A genome. Some mutants occurred more than once,

TABLE 2
Stx2A mutants (Y = yes; N = no;
s.d. = site-directed)

| Stx2 mutants | AA change | Cytotoxicity | Depurination (% wt) |
|---|---|---|---|
| | wild type (wt) | Y | 100 |
| | Group I | | |
| NT2104 | R21W | Y | 100 |
| NT2106 (s.d.) | N75A | N | 0 |
| NT2109 (s.d.) | Y77A | N | 0 |
| NT2110 | R119C | Y | 74 |
| NT2126 | S134L | Y | |
| NT2105 | S137L | | |
| NT1057 (s.d.) | E167A | N | 100 |
| NT2108 (s.d.) | R170A | N | 50 |
| NT1050 | R170H | N | |
| NT2118 | R170S | | 100 |
| NT2119 | R170P | | 100 |
| | Group II | | |
| NT2103 | R55*/R247H | N | |
| NT2111 | D111N/Q180* | N | |
| NT2122 | V136I/R204* | | |
| NT2115 (s.d.) | V235* | N | 30 |
| NT2128 (s.d.) | A236* | | |
| NT2129 (s.d.) | V237* | | |
| NT2130 (s.d.) | I238* | | |
| NT2117 (s.d.) | L239* | N | 25 |
| NT2124 (s.d.) | N240* | Y | 50 |
| NT2113 (s.d.) | C241* | Y | 40 |
| | Group III | | |
| NT2120 | V52M/G220A | | |
| NT2102 | S25L/A188V | Y | 100 |
| NT2125 (s.d.) | E167A/R170A | N | 40 |
| NT2114 | E167K/R176K | N | 0 |
| NT2123 | G221E/A282V | | |
| NT2107 | S246F/I291V | | 100 |

### Example 7

### Stx2 Mutants are not Toxic to Yeast Cells

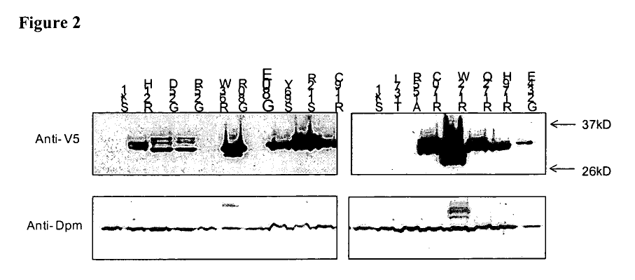

The growth inhibition of yeast cells by Stx2A single point mutants was determined by the viability assay. Yeast cells expressing wt Stx2A or the mutants were plated on SD-Ura/glucose medium after induction in galactose for 0 and 10 hours. Upon induction, wt Stx2A reduced the viability of yeast cells by at least 3 logs at 10 h (FIG. 4). Viability assay showed that the cytotoxicity of some point mutants including N75A, E167A, R170A and R170H was greatly reduced because the yeast cells harboring these mutants grew as well as those cells harboring the vector control at 10 h post-induction. E167 and R170 are the two critical amino acids in the active site of Stx2A. It has been shown that when both of them are mutated to alanine, Stx2A losses its depurination ability completely. Our results showed indeed E167A/R170A yeast cells were as viable as yeast cells transformed with the vector only. Another double mutant around the active site, E167K/R176K, was also shown to be nontoxic to yeast cells. Premature stop codon mutations at R55 and Q180 resulted in complete abolishment of Stx2A cytotoxicity.

### Example 8

### Expression of Wild-Type (wt) and Mutant Stx2 in Yeast

Immunoblot analysis with anti-V5 monoclonal antibody (Invitrogen) was used to examine the protein expression of wt Stx2A and its mutants. Total membrane fractions were separated from the cytosolic fractions of yeast cells harboring wt Stx2A and mutants. Our results have shown that as Stx1A, wt Stx2A was hardly detectable in the membrane fraction. Higher expression of mutants R21W, Y77A, E167K/R176K and R170S can be detected on the membrane fractions.

### Example 9

### Depurination of Stx2 Mutants

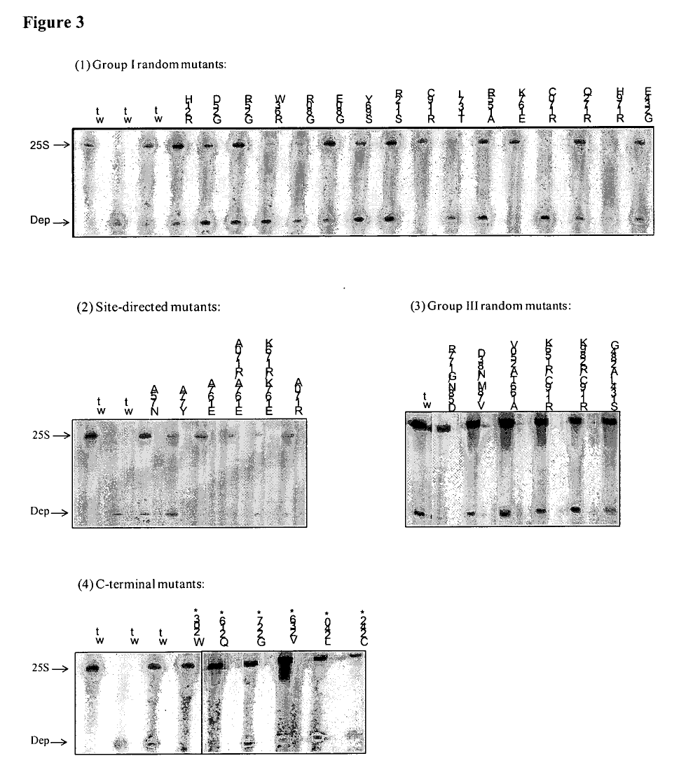

Depurination assay results (FIG. 5) on some of the mutants showed that although E167A, R170A, E167A/R170A and V235* are non-toxic (FIG. 4), they still depurinate at a lower level compared to the wt Stx1A. This data indicate that the cytotoxicity of Stx2A is not the sole result of its depurinating capability, a phenomenon that have been observed by us on PAP, RTA and Stx1A. Mutant E167K/R176K is non-toxic and non-depurinating as its counterpart of Stx1A.

### Example 10

### Interaction Between Stx1A, Stx2A and L3Δ

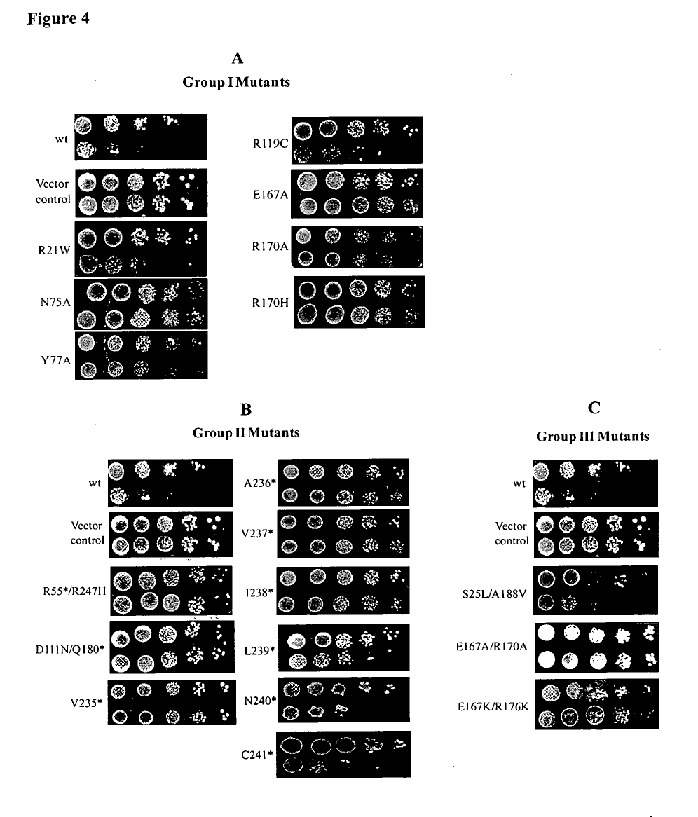

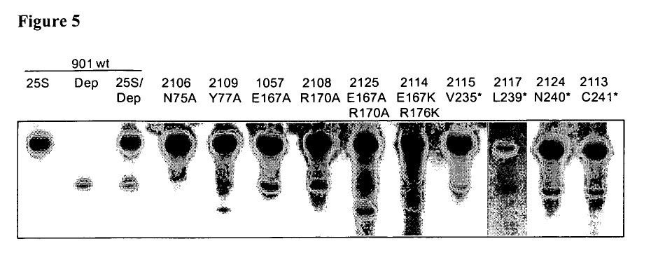

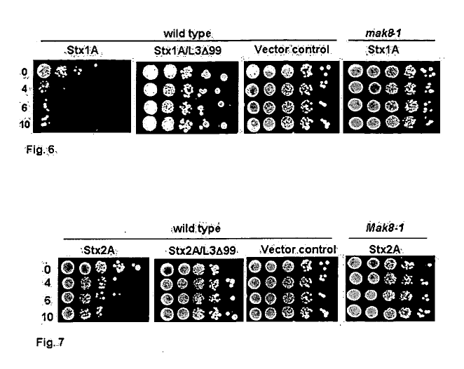

Yeast cells were used as a model system to examine the ribosome interactions of Stx1A and Stx2A and demonstrated that co-expression of a truncated form of yeast ribosomal protein L3 (L3Δ), corresponding to the first 99 amino acids of L3 overcomes the cytotoxicity of Stx1A and Stx2A in yeast (FIGS. 6 and 7). To assess the level of rRNA depurination, total RNA was extracted from the co-transformants and analyzed by the dual-primer extension analysis six hours after induction of Stx1A and Stx2A expression. As shown in FIG. 8, ribosome depurination was either reduced or completely inhibited in co-transformants containing Stx1A or Stx2A and L3Δ, compared to cells expressing Stx1A or Stx2A alone. These results demonstrated that co-expression of L3Δ inhibits the cytotoxicity of Stx1A and Stx2A in yeast by preventing ribosome depurination.

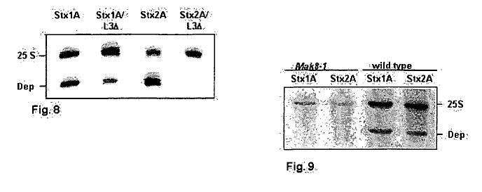

To determine if binding to ribosomal protein L3 is critical for ribosome depurination, we examined the cytotoxicity of Stx1A and Stx2A in the mak8-1 mutant, which contains two point mutations (W255C and P257T) in the ribosomal protein L3. As shown in FIGS. 6 and 7 (right panel), expression of Stx1A or Stx2A did not inhibit the growth of yeast cells harboring the mak8-1 allele of RPL3. In contrast, growth of isogenic RPL3 strains was inhibited when expression of Stx1A or Stx2A was induced. Primer extension analysis indicated that ribosomes were not depurinated in mak8-1 cells expressing Stx1A or Stx2A (FIG. 9). These results provided the first evidence that ribosomal protein L3 is critical for ribosome depurination by Stx1A and Stx2A.

To examine ribosome association of Stx1A and Stx2A in wild type yeast cells, we constructed V5 epitope tagged Stx1A and Stx2A and used differential centrifugation to isolate the membrane (P18), ribosome (P100) and the cytosolic (S100) fractions six hours after induction. Immunoblot analysis of these fractions using anti-V5, indicated that Stx1A and Stx2A are primarily associated with the membrane and the ribosome fractions.

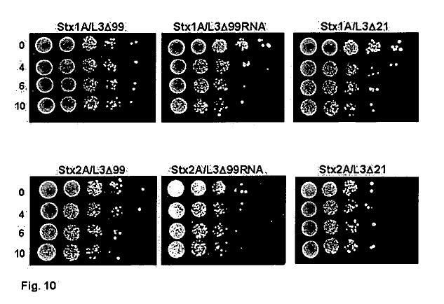

To determine if L3Δ protein or RNA expression is critical for resistance to Stx1A and Stx2A, we mutated the two methionines in the sequence of L3Δ to cysteines (M1C and M53C) and cloned the modified construct into the pYES2.1 vector (L3ΔRNA). Expression analysis using anti-V5 demonstrated that L3Δ protein is not expressed from this construct (L3ΔRNA). The L3ΔRNA, which did not produce the L3Δ peptide, did not inhibit the cytotoxicity of Stx1A in yeast (FIG. 10), indicating that expression of the L3Δ peptide is critical. To identify the minimal L3Δ sequence that can overcome the cytotoxicity of Stx1A and Stx2A, we made deletions from the C-terminus of L3Δ. One of these deletions, which corresponded to the first 21 amino acids of L3 (L3Δ21) could partially overcome the cytotoxicity of Stx1A and Stx2A in yeast (FIG. 10), indicating that the highly conserved first 21 amino acids of L3Δ are critical.

### Example 11

### Interaction Between Stx1A, Stx2A and L3Δ in Vero Cells

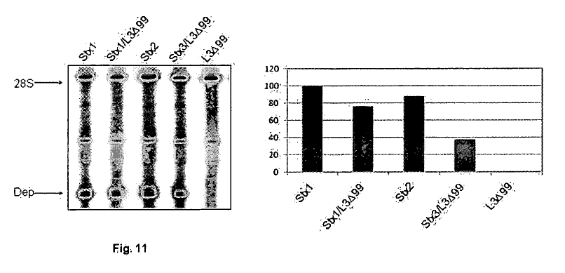

Vero (African green monkey) cells that are sensitive to the cytotoxic effects of Stx1 and Stx2 were established to examine the ability of L3Δ to eliminate these effects. Holotoxins Stx1 and Stx2 (provided by Tufts) were co-transfected by Turbofect™ (Fermentas) with L3Δ99 cloned into pcDNA3.1(+) (Invitrogen). 24 hr post transfection, total RNAs were isolated and subjected to primer extension with dual primers (28S and Dep) designed against human 28S rRNA. As shown in FIG. 11, the depurination of rRNA was reduced by 24% when Vero cells were co-transfected with L3Δ99 compared to the cells transformed with holo-Stx1. FIG. 12 also shows that L3Δ99 reduced the depurination effect of holo-Stx2 by 50%.

All publications cited in the specification, both patent publications and non-patent publications are indicative of the level of skill of those skilled in the art to which this invention pertains. Any publication not already incorporated by reference herein is herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

### INDUSTRIAL APPLICABILITY

The present invention has industrial applicability in medicine including the prevention and treatment of infection mediated by Shiga toxin and Shiga-like toxin producing microorganisms. Therefore, it has applicability in treating food poisoning and as a defense against bioterrorism.

1. An isolated nontoxic mutant comprising an Al subunit of Shiga-like toxin, wherein said mutant differs from a wild-type subunit Al of Shiga-like toxin 1 (Stx1), designated by SEQ ID NO:6, in terms of one or more amino acid substitutions, or wherein said mutant differs from a wild-type subunit A1 of Shiga-like toxin 2 (Stx2), designated as SEQ ID NO:8, in terms of one or more amino acid substitutions, except (1-297, E167/R170A), and/or which lacks from about 9 to 193 C-terminal residues thereof.

2. The isolated nontoxic mutant of claim 1, which is a mutant of Stx1.

3. The isolated nontoxic mutant of claim 2, which differs from SEQ ID NO:6 in terms of a single amino acid substitution.

4. The isolated nontoxic mutant of claim 3, which is Stx1 (1-251, G25D), Stx1 (1-251, G25R), Stx1 (1-251, N75A), Stx1 (1-251, Y77A), Stx1 (1-251,G80E), Stx1 (1-251, G80R), Stx1 (1-251, S96Y), Stx (1-251, A155R), Stx1 (1-251, E167A), Stx1 (1-251, E167K) and Stx1 (1-251, R170A).

5. The isolated nontoxic mutant of claim 2, which differs from SEQ ID NO:6 in terms of at least two amino acid substitutions.

6. The isolated nontoxic mutant of claim 5, which is Stx1 (1-251, D58N, G177R), Stx1 (1-251, V78M, N83D), Stx1 (1-251, A166T, A250V), Stx1 (1-251, R119C, R289K), Stx1 (1-251, S134L, A251G), Stx1 (1-251, E167A, R170A) or Stx1 (1-251, E167K, R176K).

7. The isolated nontoxic mutant of claim 1, which is a mutant of Stx2.

8. The isolated nontoxic mutant of claim 7, which differs from SEQ ID NO:8 in terms of a single amino acid substitution.

9. The isolated nontoxic mutant of claim 8, which is Stx2 (1-247, N75A), Stx2 (1-247, Y77A), Stx2 (1-247, E167A), Stx2 (1-247, R170A) or Stx2 (1-247, R170E).

10. The isolated nontoxic mutant of claim 7, which differs from SEQ ID NO:8 in terms of at least two amino acid substitutions.

11. The isolated nontoxic mutant of claim 10, which is Stx2 (1-297, E167K, R176K).

12. The isolated nontoxic mutant of claim 7, which differs from SEQ ID NO:8 in that it lacks from 9 to about 193 C-terminal residues thereof.

13. The isolated nontoxic mutant of claim 12, which is Stx2 (1-54), Stx2 (1-234) or Stx2 (1-238).

14. The isolated nontoxic mutant of claim 7, which differs from SEQ ID NO:8 in terms of one or more amino acid substitutions and that it lacks from 9 to about 193 C-terminal residues thereof.

15. The isolated nontoxic mutant of claim 14, which is Stx2 (1-179, D111N).

16. An isolated nucleic acid encoding the nontoxic mutant of claim 1.

17. A vector comprising the nucleic acid encoding the nontoxic mutant of claim 1, in operable association with a promoter functional in a predetermined cell.

18. A non-human host containing the vector of claim 17.

19. The non-human host of claim 14, which is E. coli.

20. A composition comprising the nontoxic mutant of claim 1, and a carrier.

21. A method of treating an individual at risk of exposure to E. coli 0157:E7 or suspected of having hemolytic euremic syndrome (HUS), comprising administering to an individual in need thereof an effective amount of a nontoxic mutant of Stx1 or Stx2, wherein said mutant comprises a non-wild-type A1 subunit.

22. A method of treating hemolytic euremic syndrome (EUS) infection comprising administering to a human in need thereof an effective amount of a nontoxic mutant of Stx1 or Stx2, wherein said mutant comprises a non-wild-type A1 subunit of Stx1 or Stx2, an effective amount of a eukaryotic L3 protein or fragment thereof containing from 21 to about 100 N-terminal amino acids, or effective amounts of both said nontoxic mutant and said L3 protein or fragment thereof.

23. A method of treating hemolytic euremic syndrome (EUS) infection comprising administering to a human in need thereof an effective amount of a eukaryotic L3 protein or fragment thereof containing from 21 to about 100 N-terminal amino acids.

24. The isolated nontoxic mutant of claim 5, wherein the at least two amino acid substitutions are E167K and R176K.

25. The isolated nontoxic mutant of claim 24, and which also lacks from about 9 to 48 C-terminal residues of the Stx1A1 subunit designated by SEQ ID NO. 6.

26. The isolated nontoxic mutant of claim 10, wherein the at least two amino acid substitutions are E167K and R176K.

27. The isolated nontoxic mutant of claim 26, and which also lacks from about 9 to 45 C-terminal residues of the Stx2A1 subunit designated by SEQ ID NO. 8.


SEQUENCE LISTING (17 sequences)

SEQ ID NO: 1 - DNA, 882 bp, Escherichia coli
  CDS (1)..(879)
aaggaatttaccttagacttctcgactgcaaagacgtatgtagattcgctgaatgtcattcgctctgcaataggtactccattacagactatttcatcaggaggtacgtctttactgatgattgatagtggctcaggggataatttgtttgcagttgatgtcagagggatagatccagaggaagggcggtttaataatctacggcttattgttgaacgaaataatttatatgtgacaggatttgttaacaggacaaataatgttttttatcgctttgctgatttttcacatgttacctttccaggtacaacagcggttacattgtctggtgacagtagctataccacgttacagcgtgttgcagggatcagtcgtacggggatgcagataaatcgccattcgttgactacttcttatctggatttaatgtcgcatagtggaacctcactgacgcagtctgtggcaagagcgatgttacggtttgttactgtgacagctgaagctttacgttttcggcaaatacagaggggatttcgtacaacactggatgatctcagtgggcgttcttatgtaatgactgctgaagatgttgatcttacattgaactggggaaggttgagtagcgtcctgcctgactatcatggacaagactctgttcgtgtaggaagaatttcttttggaagcattaatgcaattctgggaagcgtggcattaatactgaattgtcatcatcatgcatcgcgagttgccagaatggcatctgatgagtttccttctatgtgtccggcagatggaagagtccgtgggattacgcacaataaaatattgtgggattcatccactctgggggcaattctgatgcgcagaactattagcagttga

SEQ ID NO: 2 - protein, 293 aa, Escherichia coli
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVARMASDEFPSMCPADGRVRGITHNKILWDSSTLGAILMRRTISS

SEQ ID NO: 3 - DNA, 894 bp, Escherichia coli
  CDS (1)..(891)
cgggagtttacgatagacttttcgacccaacaaagttatgtctcttcgttaaatagtatacggacagagatatcgacccctcttgaacatatatctcaggggaccacatcggtgtctgttattaaccacaccccaccgggcagttattttgctgtggatatacgagggcttgatgtctatcaggcgcgttttgaccatcttcgtctgattattgagcaaaataatttatatgtggccgggttcgttaatacggcaacaaatactttctaccgtttttcagattttacacatatatcagtgcccggtgtgacaacggtttccatgacaacggacagcagttataccactctgcaacgtgtcgcagcgctggaacgttccggaatgcaaatcagtcgtcactcactggtttcatcatatctggcgttaatggagttcagtggtaatacaatgaccagagatgcatccagagcagttctgcgttttgtcactgtcacagcagaagccttacgcttcaggcagatacagagagaatttcgtcaggcactgtctgaaactgctcctgtgtatacgatgacgccgggagacgtggacctcactctgaactgggggcgaatcagcaatgtgcttccggagtatcggggagaggatggtgtcagagtggggagaatatcctttaataatatatcagcgatactggggactgtggccgttatactgaattgccatcatcagggggcgcgttctgttcgcgccgtgaatgaagagagtcaaccagaatgtcagataactggcgacaggcctgttataaaaataaacaatacattatgggaaagtaatacagctgcagcgtttctgaacagaaagtcacagtttttatatacaacgggtaaataa

SEQ ID NO: 4 - protein, 297 aa, Escherichia coli
REFTIDFSTQQSYVSSLNSIRTEISTPLEHISQGTTSVSVINHTPPGSYFAVDIRGLDVYQARFDHLRLIIEQNNLYVAGFVNTATNTFYRFSDFTHISVPGVTTVSMTTDSSYTTLQRVAALERSGMQISRHSLVSSYLALMEFSGNTMTRDASRAVLRFVTVTAEALRFRQIQREFRQALSETAPVYTMTPGDVDLTLNWGRISNVLPEYRGEDGVRVGRISFNNISAILGTVAVILNCHHQGARSVRAVNEESQPECQITGDRPVIKINNTLWESNTAAAFLNRKSQFLYTTGK

SEQ ID NO: 5 - DNA, 753 bp, Escherichia coli
  CDS (1)..(753)
aaggaatttaccttagacttctcgactgcaaagacgtatgtagattcgctgaatgtcattcgctctgcaataggtactccattacagactatttcatcaggaggtacgtctttactgatgattgatagtggctcaggggataatttgtttgcagttgatgtcagagggatagatccagaggaagggcggtttaataatctacggcttattgttgaacgaaataatttatatgtgacaggatttgttaacaggacaaataatgttttttatcgctttgctgatttttcacatgttacctttccaggtacaacagcggttacattgtctggtgacagtagctataccacgttacagcgtgttgcagggatcagtcgtacggggatgcagataaatcgccattcgttgactacttcttatctggatttaatgtcgcatagtggaacctcactgacgcagtctgtggcaagagcgatgttacggtttgttactgtgacagctgaagctttacgttttcggcaaatacagaggggatttcgtacaacactggatgatctcagtgggcgttcttatgtaatgactgctgaagatgttgatcttacattgaactggggaaggttgagtagcgtcctgcctgactatcatggacaagactctgttcgtgtaggaagaatttcttttggaagcattaatgcaattctgggaagcgtggcattaatactgaattgtcatcatcatgcatcgcgagttgccaga

SEQ ID NO: 6 - protein, 251 aa, Escherichia coli
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVAR

SEQ ID NO: 7 - DNA, 741 bp, Escherichia coli
  CDS (1)..(741)
cgggagtttacgatagacttttcgacccaacaaagttatgtctcttcgttaaatagtatacggacagagatatcgacccctcttgaacatatatctcaggggaccacatcggtgtctgttattaaccacaccccaccgggcagttattttgctgtggatatacgagggcttgatgtctatcaggcgcgttttgaccatcttcgtctgattattgagcaaaataatttatatgtggccgggttcgttaatacggcaacaaatactttctaccgtttttcagattttacacatatatcagtgcccggtgtgacaacggtttccatgacaacggacagcagttataccactctgcaacgtgtcgcagcgctggaacgttccggaatgcaaatcagtcgtcactcactggtttcatcatatctggcgttaatggagttcagtggtaatacaatgaccagagatgcatccagagcagttctgcgttttgtcactgtcacagcagaagccttacgcttcaggcagatacagagagaatttcgtcaggcactgtctgaaactgctcctgtgtatacgatgacgccgggagacgtggacctcactctgaactgggggcgaatcagcaatgtgcttccggagtatcggggagaggatggtgtcagagtggggagaatatcctttaataatatatcagcgatactggggactgtggccgttatactgaattgccatcatcagggggcgcgt

SEQ ID NO: 8 - protein, 247 aa, Escherichia coli
REFTIDFSTQQSYVSSLNSIRTEISTPLEHISQGTTSVSVINHTPPGSYFAVDIRGLDVYQARFDHLRLIIEQNNLYVAGFVNTATNTFYRFSDFTHISVPGVTTVSMTTDSSYTTLQRVAALERSGMQISRHSLVSSYLALMEFSGNTMTRDASRAVLRFVTVTAEALRFRQIQREFRQALSETAPVYTMTPGDVDLTLNWGRISNVLPEYRGEDGVRVGRISFNNISAILGTVAVILNCHHQGAR

SEQ ID NO: 9 - DNA, 1164 bp, Saccharomyces cerevisiae
  CDS (1)..(1161)
atgtctcacagaaagtacgaagcaccacgtcacggtcatttaggtttcttgccaagaaagagagctgcctccatcagagctagagttaaggcttttccaaaggatgacagatccaagccagttgctctaacttccttcttgggttacaaggctggtatgaccaccattgtcagagatttggacagaccaggttctaagttccacaagcgtgaagttgtcgaagctgtcaccgttgttgacactccaccagttgtcgttgttggtgttgtcggttacgtcgaaaccccaagaggtttgagatctttgaccaccgtctgggctgaacatttgtctgacgaagtcaagagaagattctacaagaactggtacaagtctaagaagaaggctttcaccaaatactctgccaagtacgctcaagatggtgctggtattgaaagagaattggctagaatcaagaagtacgcttccgtcgtcagagttttggtccacactcaaatcagaaagactccattggctcaaaagaaggctcatttggctgaaatccaattgaacggtggttccatctctgaaaaggttgactgggctcgtgaacatttcgaaaagactgttgctgtcgacagcgtttttgaacaaaacgaaatgattgacgctattgctgtcaccaagggtcacggtttcgaaggtgttacccacagatggggtactaagaaattgccaagaaagactcacagaggtctaagaaaggttgcttgtattggtgcttggcatccagcccacgttatgtggagtgttgccagagctggtcaaagaggttaccattccagaacctccattaaccacaagatttacagagtcggtaagggtgatgatgaagctaacggtgctaccagcttcgacagaaccaagaagactattaccccaatgggtggtttcgtccactacggtgaaattaagaacgacttcatcatggttaaaggttgtatcccaggtaacagaaagagaattgttactttgagaaagtctttgtacaccaacacttctagaaaggctttggaagaagtcagcttgaagtggattgacactgcttctaagttcggtaagggtagattccaaaccccagctgaaaagcatgctttcatgggtactttgaagaaggacttgtaa

SEQ ID NO: 10 - protein, 99 aa, Saccharomyces cerevisiae
MSHRKYEAPRHGHLGFLPRKRAASIRARVKAFPKDDRSKPVALTSFLGYKAGMTTIVRDLDRPGSKFHKREVVEAVTVVDTPPVVVVGVVGYVETPRGL

SEQ ID NO: 11 - DNA, 298 bp, Saccharomyces cerevisiae
atgtctcacagaaagtacgaagcaccacgtcacggtcatttaggtttcttgccaagaaagagagctgcctccatcagagctagagttaaggcttttccaaaggatgacagatccaagccagttgctctaacttccttcttgggttacaaggctggtatgaccaccattgtcagagatttggacagaccaggttctaagttccacaagcgtgaagttgtcgaagctgtcaccgttgttgacactccaccagttgtcgttgttggtgttgtcggttacgtcgaaaccccaagaggtttga

SEQ ID NO: 12 - protein, 21 aa, Saccharomyces cerevisiae
MSHRKYEAPRHGHLGFLPRKR

SEQ ID NO: 13 - protein, 22 aa, Saccharomyces cerevisiae
MSHRKYEAPRHGHLGFLPRKRA

SEQ ID NO: 14 - protein, 23 aa, Saccharomyces cerevisiae
MSHRKYEAPRHGHLGFLPRKRAA

SEQ ID NO: 15 - protein, 24 aa, Saccharomyces cerevisiae
MSHRKYEAPRHGHLGFLPRKRAAS

SEQ ID NO: 16 - protein, 25 aa, Saccharomyces cerevisiae
MSHRKYEAPRHGHLGFLPRKRAASI

SEQ ID NO: 17 - protein, 387 aa, Saccharomyces cerevisiae
MSHRKYEAPRHGHLGFLPRKRAASIRARVKAFPKDDRSKPVALTSFLGYKAGMTTIVRDLDRPGSKFHKREVVEAVTVVDTPPVVVVGVVGYVETPRGLRSLTTVWAEHLSDEVKRRFYKNWYKSKKKAFTKYSAKYAQDGAGIERELARIKKYASVVRVLVHTQIRKTPLAQKKAHLAEIQLNGGSISEKVDWAREHFEKTVAVDSVFEQNEMIDAIAVTKGHGFEGVTHRWGTKKLPRKTHRGLRKVACIGAWHPAHVMWSVARAGQRGYHSRTSINHKIYRVGKGDDEANGATSFDRTKKTITPMGGFVHYGEIKNDFIMVKGCIPGNRKRIVTLRKSLYTNTSRKALEEVSLKWIDTASKFGKGRFQTPAEKHAFMGTLKKDL